SEQUENCE LISTING (61 sequences)

SEQ ID NO: 1 - protein, 281 aa, unidentified
MEERNDWHFNIGAMYEIENVEGYGEDMDGLAEPSVYFNAANGPWRIALAYYQEGPVDYSAGKRGTWFDRPELEVHYQFLENDDFSFGLTGGFRNYGYHYVDEPGKDTANMQRWKIAPDWDVKLTDDLRFNGWLSMYKFANDLNTTGYADTRVETETGLQYTFNETVALRVNYYLERGFNMDDSRNNGEFSTQEIRAYLPLTLGNHSVTPYTRIGLDRWSNWDWQDDIEREGHDFNRVGLFYGYDFQNGLSVSLEYAFEWQDHDEGDSDKFHYAGVGVNYSF

SEQ ID NO: 2 - protein, 280 aa, unidentified
MEERNDWHFNIGAMYEIENVEGYGEDMDGLAEPSVYFNAANGPWRIALAYYQEGPVDYSAGKRGTWFDRPELEVHYQFLENDDFSFGLTGGFRNYGYHYVDEPGKDTANMQRWKIAPDWDVKLTDDLRFNGWLSMYKFANDLNTTGYADTRVETETGLQYTFNETVALRVNYYLERGFNMDDSRNNGEFSTQEIRAYLPLTLGNHSVTPYTRIGLDRWSNWDWQDDIEREGHDFNRVGLFYGYDFQNGLSVSLEYAFEWQDHDEGDSDKFHYAGVGVNYS

SEQ ID NO: 3 - protein, 304 aa, unidentified
MSTLLRSAALVLCAGVSCAQATEKATQWEFNIGAMYEIENVEGQGEDKDGLYEPSVWFNATWDAWTISLAMYQEGPVDYSSMTRGTYFDRPEFELRYRFIGTDDFTLGLTGGFRNYGYHFKDEHGAKDGSANMQRYKIQPDWDIKLTDDWRFGGWFAMYQFANDLEKTGYADSRVETETGFTWTLNETFAAKVNYYLERGFNMDGSRNNGEFSTQEIRAYLPISLGQTTLTPYTRLGLDRWSNWDWQDDPEREGHDFNRLGMLYAYDFNNGLSMTLEYAYEWENHDEGESDRFHYAGVGVNYAF

SEQ ID NO: 4 - protein, 304 aa, unidentified
MSTLLRSAALVLCAGVSCAHATESAKHWEFNIGAMYEIENVEGQGDDKDGLYEPSVWFNATWDAWTLSLAMYQEGPVDYSSMTRGTYFDRPEFELRYRFIGTDDFTLGLTGGFRNYGYHFKDEHGAKDGSANMQRYKIQPDWDIKLSDDWRFGGWFAMYQFANDLEKTGYADSRVETETGFTWTINETFSAKVNYYLERGFNMDSSRNNGEFSTQEIRAYLPVSLGQTTLTPYTRLGLDRWSNWDWQDDPDREGHDFNRLGLLYAYDFNNGLSMTLEYAYEWENHDEGDSDRFHYAGVGVNYAF

SEQ ID NO: 5 - protein, 304 aa, unidentified
MSTLLKSAALVLCAGVSCAHATETAKQWEFNIGAMYEIENVEGQGEDKDGLYEPSVWFNATWDAWTISLAMYQEGPVDYSSMTRGTYFDRPEFELRYRFIGTDDFTFGLTGGFRNYGYHFKDEHGARDGSANMQRYKIQPDWDIKLTDDWRFGGWFAMYQFANDLEKTGYSDSRVETETGFTWTINDTFAAKVNYYLERGFNMDGSRNNGEFSTQEIRAYLPISLGQTTLTPYTRLGLDRWSNWDWQDDPEREGHDFNRLGMQYAYDFNNGLSMTLEYAYEWENHDEGKNDRFHYAGVGVNYAF

SEQ ID NO: 6 - protein, 304 aa, unidentified
MSTLLKSAALVLCAGVSCAQATETAKQWEFNIGAMYEIENVEGQGEDKDGLYEPSVWFNATWDAWTFSLAMYQEGTVEYSSMTRGSYFDRPEFELRYRFIGTDDLTLGLTGGFRNYGYHFKDEHGAKDGSANMQRYKIQPDWDVKLTDDWRFSGWLAMYQFANDLEKTGYADSRVETETGFTWTINNIFSAKINYYLERGFNMDGSRNNGEFATQEIRAYLPVSMGQTTLTPYTRLGLDRWSNWDWQDDPSREGHDFNRLGLLYAYDFNNGLSMTLEYAYEWQNHDEGKNDRFHYAGVGVNYAF

SEQ ID NO: 7 - protein, 304 aa, unidentified
MSTLLKSAALVLCAGVSYAQASETTKQWEFNIGAMYEIENVEGQGDDKDGLYEPSVWFNATWDAWTLSLAMYQEGPVDYSSMTRGTYFDRPEFELRYRFIGTDDFTFGLTGGFRNYGYHFKDEHGAKDGSANMQRYKIQPDWDIKLTDDWRFGGWFAMYQFANDLEKTGYADSRVETETGFTWTINETFAAKVNYYLERGFNMDRSRNNGEFSTQEIRAYLPISLGQTTLTPYTRLGLDRWSNWDWQDDPEREGHDFNRLGLLYAYDFNNGLSMTLEYAYEWENHDEGESDRFHYAGVGVNYAF

SEQ ID NO: 8 - protein, 301 aa, unidentified
MKTLLSSSALLICAGMACAQAADNKDWHFNIGAMYEIENVEGYGEDMDGLAEPSVYFNASNGPWRISLAYYQEGPVDYSAGKRGTWFDRPELEVHYQILESDDFSFGLTGGFRNYGYHYVNEAGKDTANMQRWKVAPDWNVKLTDDLRFSGWLAMYQFVNDLTTTGYSDSRVESETGLNYTFNETVGLTVNYYLERGFNLAEHRNNGEFSTQEIRAYLPISLGNTTLTPYTRIGLDRWSNWDWRDDPEREGHDFNRLGLQYAYDFQNGVSMTLEYAYEWEDHDEGDSDRFHYAGIGVNYAF

SEQ ID NO: 9 - protein, 301 aa, unidentified
MKTLLSSTALLMCAGMACAQAAENNDWHFNVGAMYEIENVEGQGEDMDGLAEPSVYFNAANGPWKISLAYYQEGPVDYSAGKRGTWFDRPELEVRYQFLESDDVNFGLTGGFRNYGYHYVNEPGKDTANMQRWKVSPDWDVKISDNVRFGGWLSLYQFVNDLSTTGYSDSRVETETGFTWNINETFSLVTNYYLERGFNIDKSRNNGEFSTQEIRAYLPVALGNTTLTPYTRIGLDRWSNWDWQDDIEREGHDFNRLGMLYAYDFQNGLSMTLEYAFEWQDHDEGERDHFHYAGVGVNYAF

SEQ ID NO: 10 - protein, 10 aa, unidentified
LREEHRDLQE

SEQ ID NO: 11 - protein, 7 aa, unidentified
RPPSAGP

SEQ ID NO: 12 - protein, 20 aa, unidentified
LKPPPQHLWRQPRTPIRIQQ

SEQ ID NO: 13 - protein, 10 aa, unidentified
EQKLISEEDL

SEQ ID NO: 14 - protein, 10 aa, unidentified
GDYKDDDDKG

SEQ ID NO: 15 - DNA, 43 bp, synthetic construct
  source 1..43: mol_type = other DNA
aaagatgacgatgataaaggaggcgaagatatggatgggctgg

SEQ ID NO: 16 - DNA, 49 bp, synthetic construct
  source 1..49: mol_type = other DNA
atcatcgtcatctttataatcaccataaccctcgacgttttctatttcg

SEQ ID NO: 17 - DNA, 39 bp, synthetic construct
  source 1..39: mol_type = other DNA
tataaagatgacgatgataaaggtggtaaacgtggaacg

SEQ ID NO: 18 - DNA, 36 bp, synthetic construct
  source 1..36: mol_type = other DNA
atcgtcatctttataatcgcccgcgctataatctac

SEQ ID NO: 19 - DNA, 39 bp, synthetic construct
  source 1..39: mol_type = other DNA
tataaagatgacgatgataaaggtggtaaagacacggcg

SEQ ID NO: 20 - DNA, 37 bp, synthetic construct
  source 1..37: mol_type = other DNA
atcgtcatctttataatcgcccggttcatcaacgtag

SEQ ID NO: 21 - DNA, 43 bp, synthetic construct
  source 1..43: mol_type = other DNA
aaagatgacgatgataaaggaggttacgctgatacccgtgtcg

SEQ ID NO: 22 - DNA, 45 bp, synthetic construct
  source 1..45: mol_type = other DNA
atcatcgtcatctttataatcaccggtagtgttcagatcgttggc

SEQ ID NO: 23 - DNA, 46 bp, synthetic construct
  source 1..46: mol_type = other DNA
taaagatgacgatgataaaggaagccgcaataacggtgagttttcc

SEQ ID NO: 24 - DNA, 45 bp, synthetic construct
  source 1..45: mol_type = other DNA
tcatcgtcatctttataatcaccgtcgtccatattgaagccgcgc

SEQ ID NO: 25 - DNA, 43 bp, synthetic construct
  source 1..43: mol_type = other DNA
tataaagatgacgatgataaaggagatgatattgaacgtgaag

SEQ ID NO: 26 - DNA, 47 bp, synthetic construct
  source 1..47: mol_type = other DNA
tttatcatcgtcatctttataatcaccctgccagtcccagttactcc

SEQ ID NO: 27 - DNA, 50 bp, synthetic construct
  source 1..50: mol_type = other DNA
tataaagatgacgatgataaaggaggcgacagtgataaattccattatgc

SEQ ID NO: 28 - DNA, 45 bp, synthetic construct
  source 1..45: mol_type = other DNA
atcatcgtcatctttataatcaccttcgtcgtgatcctgccactc

SEQ ID NO: 29 - DNA, 37 bp, synthetic construct
  source 1..37: mol_type = other DNA
gaagcgtggctgggcgcgcgtggtgatattgaacgtg

SEQ ID NO: 30 - DNA, 37 bp, synthetic construct
  source 1..37: mol_type = other DNA
gcccagccacgcttccacatcgccctgccagtcccag

SEQ ID NO: 31 - DNA, 36 bp, synthetic construct
  source 1..36: mol_type = other DNA
gtatgaacgtgtgtgatgatattgaacgtgaaggcc

SEQ ID NO: 32 - DNA, 38 bp, synthetic construct
  source 1..38: mol_type = other DNA
cacgttcatacagatctgccagtcccagttactccagc

SEQ ID NO: 33 - DNA, 68 bp, synthetic construct
  source 1..68: mol_type = other DNA
tgtatgaacatctgttggactggagaaacgcaggatgatattgaacgtgaaggccatgattttaaccg

SEQ ID NO: 34 - DNA, 65 bp, synthetic construct
  source 1..65: mol_type = other DNA
gtccaacagatgttcatacatatgagcacagtctgccagtcccagttactccagcgatccagccc

SEQ ID NO: 35 - DNA, 46 bp, synthetic construct
  source 1..46: mol_type = other DNA
tgtatgaacatttgttggaccgatgatattgaacgtgaaggccatg

SEQ ID NO: 36 - DNA, 53 bp, synthetic construct
  source 1..53: mol_type = other DNA
catttgtatgaacatttgttggaccggagatgatattgaacgtgaaggccatg

SEQ ID NO: 37 - DNA, 53 bp, synthetic construct
  source 1..53: mol_type = other DNA
ccaacaaatgttcatacaaatgagcacaccctgccagtcccagttactccagc

SEQ ID NO: 38 - DNA, 45 bp, synthetic construct
  source 1..45: mol_type = other DNA
tttgtcagaatgtgtgttattacgatgatattgaacgtgaaggcc

SEQ ID NO: 39 - DNA, 47 bp, synthetic construct
  source 1..47: mol_type = other DNA
acacacattctgacaaatttcgagctgccagtcccagttactccagc

SEQ ID NO: 40 - DNA, 52 bp, synthetic construct
  source 1..52: mol_type = other DNA
atttgtcagaatgtgtgttattacggagatgatattgaacgtgaaggccatg

SEQ ID NO: 41 - DNA, 50 bp, synthetic construct
  source 1..50: mol_type = other DNA
acacacattctgacaaatttcgagaccctgccagtcccagttactccagc

SEQ ID NO: 42 - DNA, 49 bp, synthetic construct
  source 1..49: mol_type = other DNA
atttgtcagaatgtgtgttattacggaggtaaagacacggcgaatatgc

SEQ ID NO: 43 - DNA, 50 bp, synthetic construct
  source 1..50: mol_type = other DNA
acacacattctgacaaatttcgagacccggttcatcaacgtagtgataac

SEQ ID NO: 44 - DNA, 32 bp, synthetic construct
  source 1..32: mol_type = other DNA
gaggaaaggaacgactggcactttaatatcgg

SEQ ID NO: 45 - DNA, 47 bp, synthetic construct
  source 1..47: mol_type = other DNA
catatgtatatctccttcttaaagttaaacaaaattatttctagagg

SEQ ID NO: 46 - protein, 9 aa, synthetic construct
DVEAWLGAR

SEQ ID NO: 47 - protein, 15 aa, synthetic construct
TVLICMNICWTGETQ

SEQ ID NO: 48 - protein, 10 aa, synthetic construct
LEICQNVCYY

SEQ ID NO: 49 - protein, 8 aa, synthetic construct
GDKDDDKG

SEQ ID NO: 50 - protein, 9 aa, synthetic construct
GDYKDDDKG

SEQ ID NO: 51 - protein, 9 aa, synthetic construct
GDYKDDDKG

SEQ ID NO: 52 - DNA, 35 bp, synthetic construct
  source 1..35: mol_type = other DNA
taaagatgacgatgataaagatgatattgaacgtg

SEQ ID NO: 53 - DNA, 37 bp, synthetic construct
  source 1..37: mol_type = other DNA
tcatcgtcatctttataatcctgccagtcccagttac

SEQ ID NO: 54 - DNA, 41 bp, synthetic construct
  source 1..41: mol_type = other DNA
ttataaagatgacgatgataaaggagatgatattgaacgtg

SEQ ID NO: 55 - DNA, 36 bp, synthetic construct
  source 1..36: mol_type = other DNA
tcgtcatctttataatctcccccaatgcgcgtatac

SEQ ID NO: 56 - DNA, 42 bp, synthetic construct
  source 1..42: mol_type = other DNA
aaagatgacgatgataaaggacgtgaaggccatgattttaac

SEQ ID NO: 57 - DNA, 40 bp, synthetic construct
  source 1..40: mol_type = other DNA
tatcatcgtcatctttataatctccccagcgatccagccc

SEQ ID NO: 58 - DNA, 39 bp, synthetic construct
  source 1..39: mol_type = other DNA
aaagatgacgatgataaaggagaaggccatgattttaac

SEQ ID NO: 59 - DNA, 39 bp, synthetic construct
  source 1..39: mol_type = other DNA
atcatcgtcatctttataatctccactccagcgatccag

SEQ ID NO: 60 - DNA, 48 bp, synthetic construct
  source 1..48: mol_type = other DNA
tataaagatgacgatgataaaggatttaaccgtgtaggtttattttac

SEQ ID NO: 61 - DNA, 37 bp, synthetic construct
  source 1..37: mol_type = other DNA
atcgtcatctttataatctccccagtcccagttactc

(19) United States
(12) Patent Application Publication
(10) Pub. No.: US 2024/0036027 A1
(43) Pub. Date: Feb. 1, 2024

(54) Nanopore Biosensors and Uses Thereof

(71) Applicant: UNIVERSITY OF MASSACHUSETTS, Boston, MA (US)

(72) Inventors: Min CHEN, Boston, MA (US); Joshua FOSTER, Boston, MA (US); Minji KIM, Boston, MA (US); Matthew MOORE, Boston, MA (US)

(21) Appl. No.: 18/338,855

(22) Filed: Jun. 21, 2023

Related U.S. Application Data
(60) Provisional application No. 63/366,786, filed on Jun. 22, 2022.

(51) Int. Cl. G01N 33/487

(52) U.S. Cl. CPC G01N 33/48721

(57) ABSTRACT

Disclosed herein is a composition that involves a nanopore disposed in a membrane preparation, wherein the nanopore has an outer membrane protein G (OmpG) having 8 to 22 β-strands connected by a plurality of flexible loops on a first side of the membrane preparation and a plurality short turns on a second side of the membrane preparation, wherein a heterologous peptide is inserted within one or more of the flexible loops. Also disclosed herein is a method of detecting binding of a ligand to a target, the method involving: exposing a nanopore composition disclosed herein to a target; assessing a gating pattern of the nanopore; and detecting binding of the target to the heterologous peptide based on the gating pattern.

### CROSS-REFERENCE TO RELATED APPLICATIONS

This application claims benefit of U.S. Provisional Application No. 63/366,786, filed Jun. 22, 2022, which is hereby incorporated herein by reference in its entirety.

### STATEMENT REGARDING FEDERALLY SPONSORED RESEARCH OR DEVELOPMENT

This invention was made with Government Support under Grant No. GM115442 awarded by the National Institutes of Health and Grant No. AFRI 2019-67021-29452 awarded by the US Department of Agriculture National Institute of Food and Agriculture. The Government has certain rights in the invention.

### SEQUENCE LISTING

This application contains a sequence listing filed in ST.26 format entitled “921301-1120 Sequence Listing” created on Jun. 21, 2023, and having 57,708 bytes. The content of the sequence listing is incorporated herein in its entirety.

### BACKGROUND

Rapid and selective detection of biomolecular indicators of disease, referred to as biomarkers, is imperative for accurate diagnostics. However, cost-effective detection of clinically relevant biomarkers which meets these criteria is difficult due to the complexity of patient samples, large repertoire of potential compounds, and the substantial, and often variable, dynamic range of biomarkers. Therefore, selective multiplex detection of independent markers to robustly and accurately determine patient disease state and inform optimal treatment avenues is highly desirable.

### SUMMARY

Disclosed herein is a composition that involves a nanopore disposed in a membrane preparation, wherein the nanopore has an outer membrane protein G (OmpG) having 14 β-strands connected by a plurality of flexible loops on a first side of the membrane preparation and a plurality short turns on a second side of the membrane preparation, wherein a heterologous peptide is inserted within one or more of the flexible loops.

In some embodiments, the heterologous peptide is inserted between two amino acids within the one or more flexible loops. In some embodiments, the heterologous peptide replaces one or more of the amino acids within the one or more flexible loops. In some embodiments, the number of amino acids replaced is the same as the number of amino acids in the heterologous peptide.

In some embodiments, the nanopore has outer membrane protein G (OmpG) of E. coli origin having fourteen β-strands connected by seven flexible loops on a first side of the membrane preparation and seven short turns on a second side of the membrane preparation.

In some embodiments, the OmpG has the amino acids sequence SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9, or a variant thereof having at least 80%, 85%, 90%, 95%, 96%, 97%, 98%, 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

In some embodiments, the OmpG has the amino acids sequence SEQ ID NO:1, wherein loop 1 comprises amino acids E16 to A32, loop 2 comprises amino acids Q52 to D68, loop 3 comprises amino acids G96 to N109, loop 4 comprises amino acids F138 to T150, loop 5 comprises amino acids E175 to I194, loop 6 comprises amino acids R212 to R236, and loop 7 comprises amino acids E258 to V275 of SEQ ID NO:1.

In some embodiments, the heterologous peptide is inserted within, replaces, or a combination thereof, amino acids E16 to A31 of SEQ ID NO:1. For example, in some embodiments, the heterologous peptide replaces 1, 2, 3, 4, 5, 6, 7, 8, 9, 10, 11, 12, 13, 14, 15, or 16 contiguous amino acids selected from E16, I17, E18, N19, V20, E21, G22, Y23, G24, E25, D26, M27, D28, G29, L30 and A31.

In some embodiments, the heterologous peptide is inserted within, replaces, or a combination thereof, amino acids Q52 to D68 of SEQ ID NO:1. For example, in some embodiments, the heterologous peptide replaces 1, 2, 3, 4, 5, 6, 7, 8, 9, 10, 11, 12, 13, 14, 15, or 16 contiguous amino acids selected from Q52, E53, G54, P55, V56, D57, Y58, S59, A60, G61, K62, R63, G64, T65, W66, F67, and D68.

In some embodiments, the heterologous peptide is inserted within, replaces, or a combination thereof, amino acids G96 to N109 of SEQ ID NO:1. For example, in some embodiments, the heterologous peptide replaces 1, 2, 3, 4, 5, 6, 7, 8, 9, 10, 11, 12, 13, 14, 15, or 16 contiguous amino acids selected from G96, Y97, H98, Y99, V100, D101, E102, P103, G104, K105, D106, T107, A108 and N109.

In some embodiments, the heterologous peptide is inserted within, replaces, or a combination thereof, amino acids F138 to T150 of SEQ ID NO:1. For example, in some embodiments, the heterologous peptide replaces 1, 2, 3, 4, 5, 6, 7, 8, 9, 10, 11, 12, 13, 14, 15, or 16 contiguous amino acids selected from F138, A139, N140, D141, L142, N143, T144, T145, G146, Y147, A158, D149 and T150.

In some embodiments, the heterologous peptide is inserted within, replaces, or a combination thereof, amino acids E175 to I194 of SEQ ID NO:1. For example, in some embodiments, the heterologous peptide replaces 1, 2, 3, 4, 5, 6, 7, 8, 9, 10, 11, 12, 13, 14, 15, or 16 contiguous amino acids selected from E175, R176, G177, F178, N179, M180, D181, D182, S183, R184, N185, N186, G187, E188, F189, S190, T191, Q192, E193 and I184.

In some embodiments, the heterologous peptide is inserted within, replaces, or a combination thereof, amino acids L213 to D228 of SEQ ID NO:1. Therefore, in some embodiments, the heterologous peptide is inserted after L215, D216, R217, W218, S219, N220, W221, D222, W223, Q224, D225, D226, I227, E228, R229, E230, G231, H232, or D233. For example, in some embodiments, the heterologous peptide replaces 1, 2, 3, 4, 5, 6, 7, 8, 9, 10, 11, 12, 13, 14, 15, or 16 contiguous amino acids selected from L213, D214, R215, W216, D217, W218, Q219, D220, D221, I222, E223, R224, E225, G226, H227, D228, F229, H230 and R231.

In some embodiments, there are 2, 3, 4, 5, 6, or 7 distinct heterologous peptides independently inserted within the one or more flexible loops.

In some embodiments, the membrane preparation has a planar lipid bilayer. For example, in some embodiments, the membrane preparation comprises a micelle, a bacterium, or a eukaryotic cell.

Also disclosed herein is a method of detecting binding of a ligand to a target, the method involving: exposing a nanopore composition disclosed herein to a target; assessing a gating pattern of the nanopore; and detecting binding of the target to the heterologous peptide based on the gating pattern.

In some embodiments, the target is a protein, a virus, a bacteria, a nucleic acid, or a mammalian cell. For example, in some embodiments, the target is an antibody or hybridoma.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. Schematic of an embodiment OmpG multiplex biosensor (PDB: 5MWV).

FIGS. 2a-2c. OmpG loops demonstrate variable peptide display efficiency. FIG. 2a) Top view of OmpG sensor, FLAG motif insertion site is indicated by colored spheres (PDB: 5MWV). FIG. 2b) Cartoon schematic of OmpG constructs displayed on E. coli outer membrane. FIG. 2c) Flow cytometry analysis of E. coli cells expressing OmpGLn-FLAG variants, data is presented as kernel density estimation of FITC-channel event counts combined from biological triplicates.

FIGS. 3a-3e. Detection of FG4R via OmpGL3-FLAG nanopore. FIG. 3a) Schematic model of OmpGL3-FLAG sensor and FG4R depicted to relative scale, loop 3 FLAG motif indicated by orange spheres. FIGS. 3b,3c,3d) Representative current traces of OmpGL3-FLAG in the FIG. 3b) absence or FIGS. 3c-3d) presence of FG4R (20 nM). Regions of the trace showing FG4R binding are colored in orange. The red and blue dotted lines represent 10 and FG4R-bound current respectively. FIG. 3e) All-points histogram of 30 second segments of OmpGL3-FLAG during 10 and FG4R bound states. Experiments were performed in 50 mM Na2HPO4 pH 6.0 buffer containing 300 mM KCl at ±50 mV. Traces were filtered using a 500 Hz lowpass digital gaussian filter.

FIGS. 4a-4h. Detection of Streptavidin by OmpGL6-SA1.1 nanopore. FIG. 4a) Schematic view of OmpGL6-SA1.1 sensor and analyte streptavidin, loop 6 SA1.1 motif indicated by blue spheres. FIGS. 4b-4c) Representative traces of OmpGL6-SA1.1 in the absence and presence of streptavidin (800 nM). The regions of the trace demonstrating streptavidin binding signals are colored in blue. FIGS. 4d-4f) Representative traces of streptavidin binding Subtypes I-Ill. FIGS. 4g-4h) Black line shows gaussian fit of τon and τoff from log transformed millisecond values (N=3, n=903). Experiments were performed in 50 mM Na2HPO4 pH 6.0 buffer containing 300 mM KCl at an applied potential of ±50 mV. Traces were filtered using a 500 Hz lowpass digital gaussian filter.

FIGS. 5a-5k. Multiplex detection of two protein analytes in a simple mixture by OmpGL3-FLAG/L6-SA1.1 nanopore FIG. 5a) Schematic view of OmpGL3-FLAG/L6-SA1.1 sensor, residues comprising the loop 3 FLAG and loop 6 SA1.1 motifs are indicated by orange and blue spheres respectively. FIGS. 5b-5d) Representative single-channel current recordings OmpGL3-FLAG/L6-SA1.1 b) basal behavior and recapitulation of FIG. 5c) FG4R (20 nM) and FIG. 5d) streptavidin (800 nM) binding signals. FIGS. 5e-5f) Representative single-channel current recording traces showing recapitulation of individual analyte signals when analytes are present in a simple mixture FIG. 5g) Concurrent observation of both binding signals. FIGS. 5h-5i) Concentration dependence of streptavidin 1/τon and 1/τoff. Error bars represent standard deviation from three independent pores. FIG. 5j-k) Gaussian fit of ton and toff values from log transformed millisecond values representing quantification of streptavidin binding events at 800 nM. OmpGL3-FLAG/L6-SA1.1 streptavidin only (N=3, n=813) and OmpGL3-FLAG/L6SA1.1 concurrent binding (N=1, n=146). Experiments were performed in 50 mM Na2HPO4 pH 6.0 buffer containing 300 mM KCl at an applied potential of ±50 mV. Traces were filtered using a 500 Hz lowpass digital gaussian filter.

FIGS. 6a-6i. Detection of streptavidin by OmpGL3/L6-SA1.1 avidity nanopore sensor. FIG. 6a) Schematic view of OmpGL3/L6-SA1.1 avidity sensor. FIG. 6b) Representative current trace in the absence of streptavidin FIG. 6c) Representative current trace and all-points histogram in the presence of streptavidin, binding signal(s) indicated in blue. All-points histogram generated from 30 second segments of indicated state. FIGS. 6d-6e) Gaussian fit of τon and τoff values derived from discrete streptavidin binding events at either 10 nM (N=7, n=258) or 50 nM (N=5, n=247). FIG. 6f) Zoomed in view of discrete bound state highlighting regions defined as transient dissociation (τ1On & τ2On) and streptavidin rebinding (τ′Off). g-h) Gaussian fit of kinetic parameters τ1&2On and τ′Off of recapture events at either 10 nM (N=3, n=902) or 50 nM (N=3, n=896). FIG. 6i) Hypothetical model of interaction between streptavidin and OmpGL3/L6-SA1.1 to explain observed bimodal distribution of recapture τOn. Experiments were performed in 50 mM Na2HPO4 pH 6.0 buffer containing 300 mM KCl at an applied potential of ±50 mV. Traces were filtered using a 500 Hz lowpass digital gaussian filter.

FIGS. 7a-7c. Comparison of OmpG^{wt} and OmpG^{L6-FLAG} sensor. FIG. 7(a) Sequence of the two OmpG constructs and the schematic representation of OmpG^{L6-FLAG} (PDB: 2IWW). FIG. 7(b) Single channel current recording of OmpG^{wt} and OmpG^{L6-FLAG} before and after target FG4R addition. The buffer was 300 mM KCl and 50 mM Na2HPo4, pH 6.0. The applied potential was +50 mV. FIG. 7(c) Cytometric results of uninduced/induced OmpG^{wt} and OmpG^{L6-FLAG} with FITC labeled FLAG antibody.

FIGS. 8a-8e. OmpG nanopores and its characteristics with and without FG4R. FIG. 8(a) Structure of OmpG^{wt} (PDB: 2IWW) and the sequences of the loop 6 of OmpG nanopore constructs. Loop 6 is colored in orange in the structure and the FLAG binding motifs are in red. Single channel recording with and without FG4R of FIG. 8(b) OmpG^{L215-FLAG}, FIG. 8(c) OmpG^{L219-FLAG} FIG. 8(d) OmpG^{N220-FLAG}, and FIG. 8(e) OmpG^{Q224-FLAG}. Target binding sinal was colored in yellow in current recording.

FIGS. 9a-9d. Detection of FLAG tag monoclonal antibody by OmpG^{Q224-FLAG} The final concentration of each antibody was 30 nM. FIG. 9(a) Current trace of OmpG^{Q224-FLAG} and its histogram. All the measurement was performed under the condition of 300 mM KCl, 50 mM Na2HPo4 pH 6.0 at 50 mV of voltage potential. FIG. 9(b) Representative current trace of FG4R (30 nM), and the binding signal was presented in yellow. FIGS. 9(c), 9(d) Same protocol used for FLAG tag monoclonal antibody OTI4C5 (30 nM, orange), and 29E4.G7 (30 nM, green).

FIGS. 10a-10b. Current recording of antibody mixtures and the schematic figures. FIG. 10(a) Current trace of OmpG^{Q224-FLAG} after adding two different monoclonal antibodies, FG4R and OTI4C5, in a single pore, and its schematic figure. The binding signal of FG4R in yellow color and that of OTI4C5 in orange color. FIG. 10(b) Detection of FLAG tag polyclonal antibody binding by OmpG^{Q222-FLAG} and its schematic figure.

FIG. 11. Topology model of OmpG (adapted from PDB: 5MWV). Black arrows indicate insertion site(s) utilized for peptide display within the respective loop: following Y23, A60, P103, T145, D182, Q224, and E264. Model sequence and residue numbering represent protein without endogenous signal sequence. β-strand residues are presented as squares, all other residues as circles. Model does not reflect shear angle and hydrogen-bonding of native structure.

FIGS. 12a-12b. OmpG loop 6 demonstrates variable display efficiency of streptavidin-binding peptides. FIG. 12a) Sequence of streptavidin-binding peptide inserted into OmpG loop 6. FIG. 12b) Flow cytometry analysis of E. coli cells expressing OmpG^{L6-streptavidin-binder} variants, 800 nM streptavidin-alexa647 labeling data is presented as kernel density estimation of APC-channel event counts.

FIGS. 13a-13I. Variable loop FLAG-display gating characteristics & Influence of FG4R dosing. Characterization of constructs FIGS. 13a-13c) OmpG^{L2-FLAG}, FIGS. 13d-13f) OmpG^{L4-FLAG}, FIGS. 13g-13i) and OmpG^{L6-FLAG}. Top view of OmpG highlighting FLAG motif insertion site via orange spheres, a representative single-channel current recording trace demonstrating basal pore gating in the presence of FG4R (80 nM), and corresponding two-dimensional contour plot of residual current (Iᵣₑₛ) and duration (Dwell) of gating spikes characterizing basal behavior. FIGS. 13j-13I) Top view of OmpG^{L3-FLAG} with FLAG motif insertion site highlighted by orange spheres, representative single-channel current recording trace demonstrating basal and FG4R-bound (20 nM) gating states, and corresponding two-dimensional contour plot of residual current (Ires) and duration (Dwell) of gating spikes. FG4R-bound state, shown in orange, exhibits a reduction in overall gating frequency but does not meaningfully alter spike Ires or duration from the basal state, leaving the reduction in current as the most effective parameter for event identification. Two-dimensional histogram plots were generated from MOASIC Adept 2-State call of gating within 20 second trace segments of the indicated construct, three segments were taken from each pore analyzed (N_{P}=Number of pores N_{G}=Number of total gating spikes from all analyzed gating events). Experiments were performed in 50 mM Na₂HPO₄ pH 6.0 buffer containing 300 mM KCl at ±50 mV. Traces were filtered using a 500 Hz lowpass digital gaussian filter.

FIGS. 14a-14d. Analyte signals are specific to corresponding loop-displayed affinity motif. FIGS. 14a-14d) Representative single-channel current recording traces of indicated OmpG constructs dosed with either FG4R (30 nM) or streptavidin (800 nM). In each case the pore was observed for at least 30 min for the occurrence of the previously reported analyte binding signal or other alteration of basal pore behavior. All tested constructs exhibited no change in gating characteristics in response to the indicated analyte addition showing that observed binding signals are specific to the analyte/loop-displayed affinity-tag pair. Experiments were performed in 50 mM Na₂HPO₄ pH 6.0 buffer containing 300 mM KCl at ±50 mV. Traces were filtered using a 500 Hz lowpass digital gaussian filter.

FIGS. 15a-15d. Representative gaussian fittings of τ_{On} and τ_{Off} from log transformed millisecond values. Extracted values were used in concentration dependence analysis of OmpG^{L3-FLAG/L6-SA1.1}, shown in FIGS. 5h-5i. n is the number of individual binding events analyzed per independent pore. R² value calculated using OriginPro2020b.

FIGS. 16a-16c. In vitro refolding analysis of constructs studied. All constructs were successfully purified from an inclusion body and subsequently refolded in OG micelles for 72 hr. All samples besides OmpG^{L1-FLAG} exhibit heat-modifiability indicating successful refolding, however this construct is successfully displayed on the cell membrane suggesting a discrepancy between in vivo and in vitro refolding. Higher molecular weight species observed in constructs containing the SA1.1 motif are likely dimers formed by intermolecular disulfide bond formation during refolding.

FIGS. 17a-17e. Detailed comparison of engineered OmpG gating behavior. FIG. 17a) OmpG^{wt} FIG. 17b) OmpG^{L3-FLAG} FIG. 17c) OmpG^{L6-SA1.1} FIG. 17d) OmpG^{L3-FLAG/L6-SA1.1} and FIG. 17e) OmpG^{L3/L6-SA1.1} showing top view of pore and representative single-channel current traces of cis pore gating behavior at +50 mV or −50 mV. Insertion site of loop-displayed motif is indicated by spheres. ‘Noisy’ behavior of each construct is demonstrated in the traces recorded at −50 mV when exposed loops are experiencing a positive voltage. Experiments were performed in 50 mM Na₂HPO₄ pH 6.0 buffer containing 300 mM KCl at ±50 mV. Traces were filtered using a 500 Hz lowpass digital gaussian filter.

FIGS. 18a-18c. Concurrent analyte binding alters streptavidin binding subtype distribution. FIG. 18a) Two-dimensional histogram plots generated from MOSAIC adept 2-state analysis of OmpG^{L3-FLAG/L6-SA1.1} gating behavior during indicated streptavidin binding subtype (N=number of pores, n=number of events, n_{G}=number of gating events within each binding event). FIG. 18b) Streptavidin binding subtype distribution generally and during concurrent FG4R/streptavidin binding. FIG. 18C) Two-dimensional histogram of MOSAIC adept 2-state analysis of OmpG^{L3-FLAG/L6-SA1.1} gating behavior during observed concurrent binding events without regard to subtype.

FIG. 19. Refolding analysis of OmpG construct studied. The constructs were purified from an inclusion body and refolded using OG for 3 days. To test refolding efficacy, refolded constructs were boiled at 95 C for 20 mins and subsequently loaded on a 12% SDS PAGE gel. All samples were refolded successfully, especially OmpG^{Q224 FLAG} construct was refolded more than 88%.

FIG. 20. Alignment of OmpG protein sequences. Shown are SEQ ID NOs:1 and 3-9.

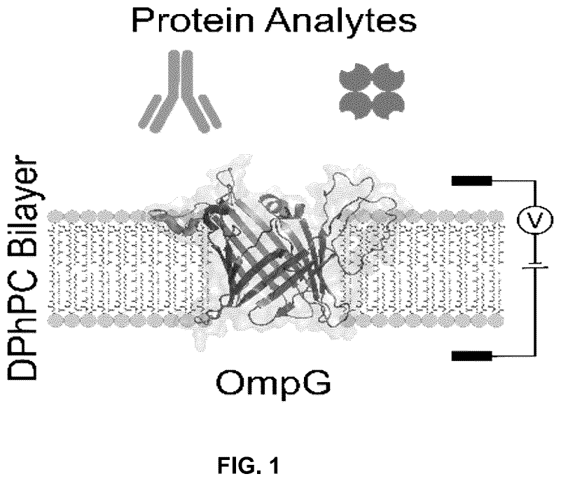

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Disclosed herein are nanopores useful as sensors for detecting intermolecular interactions. In some embodiments, nanopore sensing is based on reading ionic current passing through an individual protein or synthetic nano-sized pore. In some embodiments, the interaction between analytes and the sensor pore alters ionic current. Accordingly, in some embodiments, information about the identity of analytes, as well as their concentrations may be gathered. In some embodiments, nanopore sensing is sensitive (nanomolar concentrations), fast (up to microsecond resolution), and without delays from mixing and diffusion (real-time). In some embodiments, molecular detection using a single nanopore works by observing modulations in ionic current flowing through the pore during an applied potential.

In some embodiments, oligomeric protein nanopores with rigid structures have been engineered that are useful for sensing a wide range of analytes including small molecules and biological species such as proteins and DNA. In some embodiments, a monomeric β-barrel porin, OmpG, was selected as a platform from which to derive a nanopore sensor. In some embodiments, OmpG is decorated with several flexible loops that move dynamically to create a distinct gating pattern when ionic current passes through the pore. In some embodiments, the gating characteristic of the loop's movement in and out of a porin is substantially altered by analyte protein binding. In some embodiments, the gating characteristics of the pore with bound targets were remarkably sensitive to molecular identity. In some embodiments, gating characteristics of the pore with bound targets provided the ability to distinguish between homologues within an antibody mixture. In some embodiments, multiple gating parameters (e.g., five parameters) are analyzed for each of a number of analytes to create a unique fingerprint for each binding molecule. In some embodiments, exploitation of gating noise as a molecular identifier is advantageous because it enables sophisticated sensor designs and applications.

In some embodiments, bacterial outer membrane porins, having robust β-barrel structures, are used as stochastic sensors based on single-molecule detection. For example, in some embodiments, OmpG's monomeric structure greatly simplifies nanopore production. In some embodiments, a monomeric porin, OmpG, is advantageous as a nanopore because appropriate modifications of the pore can be easily achieved by mutagenesis. In some embodiments, gating of a nanopore causes transient current blockades in single-channel recordings that may interfere with analyte detection. In some embodiments, binding of an analyte to a nanopore partially blocks the flow of current and provides information about a molecules size, concentration and affinity. In some embodiments, direct protein detection with nanopores does not involve transmitting a binding signal from solution to a pore interior.

In some embodiments, methods are provided herein that use loop dynamics to detect protein interactions. In some embodiments, the nanopore is a monomeric protein nanopore. In certain embodiments, the nanopore comprises a plurality of β-strands connected by a plurality of flexible loops of a first side of the membrane preparation and a plurality of short turns on a second side of the membrane preparation. In some embodiments, the nanopore comprises 8 to 22 β-strands connected by flexible loops on a first side of the membrane preparation and a plurality of short turns on a second side of the membrane preparation. In certain embodiments, the nanopore comprises 14 β-strands connected by seven flexible loops on a first side of the membrane preparation and seven short turns on a second side of the membrane preparation. In some embodiments, the nanopore comprises on the first side an opening in a range of 6 to 10 Å in diameter and an opening on the second side in a range of 12 to 16 Å. In certain embodiments, a nanopore has a stabilizing mutation in a beta-barrel region. In certain embodiments, at least one of the flexible loops of the nanopore is stabilized to reduce gating. In certain embodiments, at least one of the flexible loops of the nanopore is mutated to increase its flexibility. In some embodiments, the nanopore is an outer membrane protein G composed of 14 β-strands connected by seven flexible loops on the extracellular side and seven short turns on the periplasmic side, and wherein loop 6 is stabilized to an open conformation to reduce gating. In some embodiments, the nanopore is an outer membrane protein G (OmpG). In certain embodiments, the OmpG is of E. coli origin. In some embodiments, the nanopore is a pore from a mitochondria membrane. In certain embodiments, the nanopore is a synthetic peptide or polymer that forms a pore in a membrane. In some embodiments, the nanopore spontaneously gates in the membrane preparation during an applied potential.

In some embodiments, a ligand may be incorporated directly into a nanopore (e.g., as an epitope in a nanopore loop). In some embodiments, a protein can be detected using a nanopore (e.g., a OmpG nanopore sensor). However, it should be appreciated that other biological substances, such as nucleic acids, protein complexes, bacteria, virus and cells might be detected using the same approach.

In some embodiments, OmpG homologs from other organisms can be used for sensing of large biological molecules. In addition, any pore-forming protein, e.g., porins from bacterial membrane and mitochondria membrane with flexible loops can be used to detect large molecules following the same methods disclosed herein. In some embodiments, synthetic molecules, e.g., synthetic peptides and polymers that form pores can be used for sensing in the same manner.

The antibody industry is continuously developing new and robust discovery platforms and novel antibody formats. In some embodiments, specific antibodies can be built on varied protein scaffolds after the introduction of an antigen binding site/interface.

In some embodiments, these scaffold proteins differ greatly in their origin, function and structure. In some embodiments, some of their antigen binding sites do not share any sequence or structural homology with natural antibodies. In

In some embodiments, the nanopore sensor involves inserting a heterologous peptide into the extracellular loops of OmpG.

In some embodiments, OmpG has an amino acid sequence:

(SEQ ID NO: 1)
MEERNDWHFNIGAMYEIENVEGYGEDMDGLAEPSVYFNAA
NGPWRIALAYYQEGPVDYSAGKRGTWFDRPELEVHYQFLE
NDDFSFGLTGGFRNYGYHYVDEPGKDTANMQRWKIAPDWD
VKLTDDLRFNGWLSMYKFANDLNTTGYADTRVETETGLQY
TFNETVALRVNYYLERGFNMDDSRNNGEFSTQEIRAYLPL
TLGNHSVTPYTRIGLDRWSNWDWQDDIEREGHDFNRVGLF
YGYDFQNGLSVSLEYAFEWQDHDEGDSDKFHYAGVGVNYS
F.
In some embodiments, OmpG has an
amino acid sequence:
(SEQ ID NO: 2)
MEERNDWHFNIGAMYEIENVEGYGEDMDGLAEPSVYFNAA
NGPWRIALAYYQEGPVDYSAGKRGTWFDRPELEVHYQFLE
NDDFSFGLTGGFRNYGYHYVDEPGKDTANMQRWKIAPDWD
VKLTDDLRFNGWLSMYKFANDLNTTGYADTRVETETGLQY
TFNETVALRVNYYLERGFNMDDSRNNGEFSTQEIRAYLPL
TLGNHSVTPYTRIGLDRWSNWDWQDDIEREGHDFNRVGLF
YGYDFQNGLSVSLEYAFEWQDHDEGDSDKFHYAGVGVNYS.

In some embodiments, loop 1 comprises amino acids 16 to 31, loop 2 comprises amino acids 52 to 68, loop 3 comprises amino acids 96 to 109, loop 4 comprises amino acids 138 to 150, loop 5 comprises amino acids 175 to 194, loop 6 comprises amino acids 212 to 236, and loop 7 comprises amino acids 258 to 275 of SEQ ID NO:1 or 2. Therefore, in some embodiments, the heterologous peptide is inserted, replaces, or a combination thereof, within loop 1, i.e. amino acids 16 to 31 of SEQ ID NO:1 or 2. Therefore, in some embodiments, the heterologous peptide is inserted, replaces, or a combination thereof, within loop 2, i.e. amino acids 52 to 68 of SEQ ID NO:1 or 2. Therefore, in some embodiments, the heterologous peptide is inserted, replaces, or a combination thereof, within loop 3, i.e. amino acids 96 to 109 of SEQ ID NO:1 or 2. Therefore, in some embodiments, the heterologous peptide is inserted, replaces, or a combination thereof, within loop 4, i.e. amino acids 138 to 150 of SEQ ID NO:1 or 2. Therefore, in some embodiments, the heterologous peptide is inserted, replaces, or a combination thereof, within loop 5, i.e. amino acids 175 to 194 of SEQ ID NO:1 or 2. Therefore, in some embodiments, the heterologous peptide is inserted, replaces, or a combination thereof, within loop 6, i.e. amino acids R212 to R236 of SEQ ID NO:1 or 2. Therefore, in some embodiments, the heterologous peptide is inserted, replaces, or a combination thereof, within loop 6, i.e. amino acids E258 to V275 of SEQ ID NO:1 or 2. Therefore, in some embodiments, the heterologous peptide is inserted, replaces, or a combination thereof, within loop 7, i.e. amino acids E258 to V275 of SEQ ID NO:1.

Therefore, in some embodiments, the heterologous peptide is inserted after R212, I213, G214, L215, D216, R217, W218, S219, N220, W221, D222, W223, Q224, D225, D226, I227, E228, R229, E230, G231, H232, D233, F234, N235, R236. Therefore, in some embodiments, the heterologous peptide replaces 1, 2, 3, 4, 5, 6, 7, 8, 9, 10, 11, 12, 13, 14, 15, or 16 contiguous amino acids selected from R212, I213, G214, L215, D216, R217, W218, S219, N220, W221, D222, W223, Q224, D225, D226, I227, E228, R229, E230, G231, H232, D233, F234, N235, and R236.

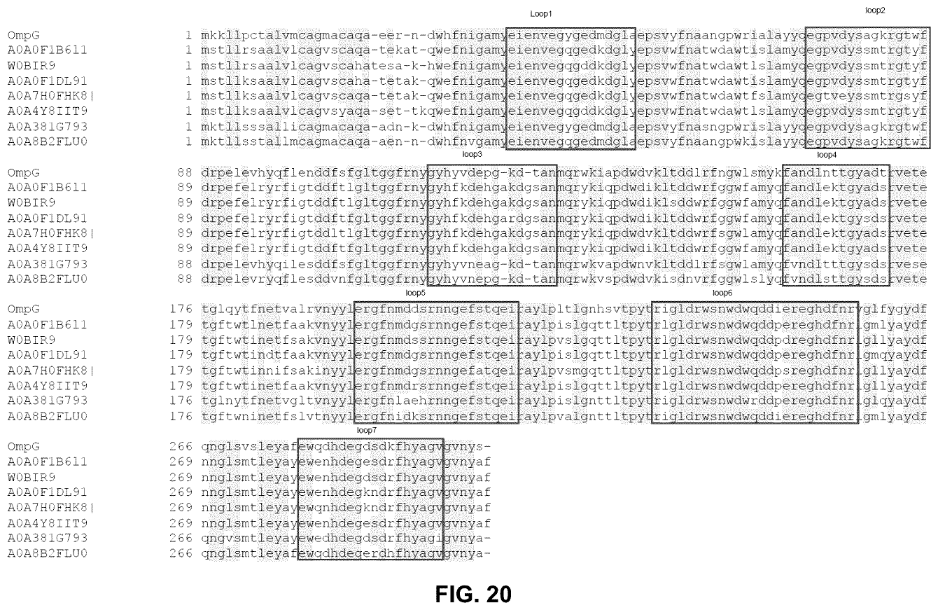

Additional OmpG sequences and their alignments are shown in FIG. 20. For example, in some embodiments, OmpG has an amino acid sequence:

(SEQ ID NO: 3, A0A0F1B611)
MSTLLRSAALVLCAGVSCAQATEKATQWEFNIGAMYEIEN
VEGQGEDKDGLYEPSVWFNATWDAWTISLAMYQEGPVDYS
SMTRGTYFDRPEFELRYRFIGTDDFTLGLTGGFRNYGYHF
KDEHGAKDGSANMQRYKIQPDWDIKLTDDWRFGGWFAMYQ
FANDLEKTGYADSRVETETGFTWTLNETFAAKVNYYLERG
FNMDGSRNNGEFSTQEIRAYLPISLGQTTLTPYTRLGLDR
WSNWDWQDDPEREGHDFNRLGMLYAYDFNNGLSMTLEYAY
EWENHDEGESDRFHYAGVGVNYAF.

In some embodiments, loop 1 comprises amino acids 37 to 52, loop 2 comprises amino acids 74 to 88, loop 3 comprises amino acids 117 to 132, loop 4 comprises amino acids 161 to 173, loop 5 comprises amino acids 198 to 217, loop 6 comprises amino acids 235 to 259, and loop 7 comprises amino acids 281 to 298 of SEQ ID NO:3. Therefore, in some embodiments, the heterologous peptide is inserted, replaces, or a combination thereof, within loop 1, i.e. amino acids 37 to 52 of SEQ ID NO:3. Therefore, in some embodiments, the heterologous peptide is inserted, replaces, or a combination thereof, within loop 2, i.e. amino acids 74 to 88 of SEQ ID NO:3. Therefore, in some embodiments, the heterologous peptide is inserted, replaces, or a combination thereof, within loop 3, i.e. amino acids 1117 to 132 of SEQ ID NO:3. Therefore, in some embodiments, the heterologous peptide is inserted, replaces, or a combination thereof, within loop 4, i.e. amino acids 161 to 173 of SEQ ID NO:3. Therefore, in some embodiments, the heterologous peptide is inserted, replaces, or a combination thereof, within loop 5, i.e. amino acids 198 to 217 of SEQ ID NO:3. Therefore, in some embodiments, the heterologous peptide is inserted, replaces, or a combination thereof, within loop 6, i.e. amino acids 235 to 259 of SEQ ID NO:3. Therefore, in some embodiments, the heterologous peptide is inserted, replaces, or a combination thereof, within loop 7, i.e. amino acids 281 to 298 of SEQ ID NO:3.

In some embodiments, OmpG has an amino acid sequence:

(SEQ ID NO: 4, W0BIR9)
MSTLLRSAALVLCAGVSCAHATESAKHWEFNIGAMYEIEN
VEGQGDDKDGLYEPSVWFNATWDAWTLSLAMYQEGPVDYS
SMTRGTYFDRPEFELRYRFIGTDDFTLGLTGGFRNYGYHF
KDEHGAKDGSANMQRYKIQPDWDIKLSDDWRFGGWFAMYQ
FANDLEKTGYADSRVETETGFTWTINETFSAKVNYYLERG
FNMDSSRNNGEFSTQEIRAYLPVSLGQTTLTPYTRLGLDR
WSNWDWQDDPDREGHDFNRLGLLYAYDFNNGLSMTLEYAY
EWENHDEGDSDRFHYAGVGVNYAF.

In some embodiments, loop 1 comprises amino acids 37 to 52, loop 2 comprises amino acids 74 to 88, loop 3 comprises amino acids 117 to 132, loop 4 comprises amino acids 161 to 173, loop 5 comprises amino acids 198 to 216, loop 6 comprises amino acids 235 to 259, and loop 7 comprises amino acids 281 to 298 of SEQ ID NO:4. Therefore, in some embodiments, the heterologous peptide is inserted, replaces, or a combination thereof, within loop 1, i.e. amino acids 37 to 52 of SEQ ID NO:4. Therefore, in some embodiments, the heterologous peptide is inserted, replaces, or a combination thereof, within loop 2, i.e. amino acids 74 to 88 of SEQ ID NO:4. Therefore, in some embodiments, the heterologous peptide is inserted, replaces, or a combination thereof, within loop 3, i.e. amino acids 117 to 132 of SEQ ID NO:4. Therefore, in some embodiments, the heterologous peptide is inserted, replaces, or a combination thereof, within loop 4, i.e. amino acids 161 to 173 of SEQ ID NO:4. Therefore, in some embodiments, the heterologous peptide is inserted, replaces, or a combination thereof, within loop 5, i.e. amino acids 198 to 216 of SEQ ID NO:4. Therefore, in some embodiments, the heterologous peptide is inserted, replaces, or a combination thereof, within loop 6, i.e. amino acids 235 to 259 of SEQ ID NO:4. Therefore, in some embodiments, the heterologous peptide is inserted, replaces, or a combination thereof, within loop 7, i.e. amino acids 281 to 298 of SEQ ID NO:4.

In some embodiments, OmpG has an amino acid sequence:

(SEQ ID NO: 5, A0A0F1DL91)
MSTLLKSAALVLCAGVSCAHATETAKQWEFNIGAMYEIEN
VEGQGEDKDGLYEPSVWFNATWDAWTISLAMYQEGPVDYS
SMTRGTYFDRPEFELRYRFIGTDDFTFGLTGGFRNYGYHF
KDEHGARDGSANMQRYKIQPDWDIKLTDDWRFGGWFAMYQ
FANDLEKTGYSDSRVETETGFTWTINDTFAAKVNYYLERG
FNMDGSRNNGEFSTQEIRAYLPISLGQTTLTPYTRLGLDR
WSNWDWQDDPEREGHDFNRLGMQYAYDFNNGLSMTLEYAY
EWENHDEGKNDRFHYAGVGVNYAF.

In some embodiments, loop 1 comprises amino acids 37 to 52, loop 2 comprises amino acids 74 to 88, loop 3 comprises amino acids 117 to 132, loop 4 comprises amino acids 161 to 173, loop 5 comprises amino acids 198 to 217, loop 6 comprises amino acids 235 to 259, and loop 7 comprises amino acids 281 to 298 of SEQ ID NO:5. Therefore, in some embodiments, the heterologous peptide is inserted, replaces, or a combination thereof, within loop 1, i.e. amino acids 37 to 52 of SEQ ID NO:5. Therefore, in some embodiments, the heterologous peptide is inserted, replaces, or a combination thereof, within loop 2, i.e. amino acids 74 to 88 of SEQ ID NO:5. Therefore, in some embodiments, the heterologous peptide is inserted, replaces, or a combination thereof, within loop 3, i.e. amino acids 117 to 132 of SEQ ID NO:5. Therefore, in some embodiments, the heterologous peptide is inserted, replaces, or a combination thereof, within loop 4, i.e. amino acids 161 to 173 of SEQ ID NO:5. Therefore, in some embodiments, the heterologous peptide is inserted, replaces, or a combination thereof, within loop 5, i.e. amino acids 198 to 217 of SEQ ID NO:5. Therefore, in some embodiments, the heterologous peptide is inserted, replaces, or a combination thereof, within loop 6, i.e. amino acids 235 to 259 of SEQ ID NO:5. Therefore, in some embodiments, the heterologous peptide is inserted, replaces, or a combination thereof, within loop 7, i.e. amino acids 281 to 298 of SEQ ID NO:5.

In some embodiments, OmpG has an amino acid sequence:

(SEQ ID NO: 6, A0A7H0FHK8)
MSTLLKSAALVLCAGVSCAQATETAKQWEFNIGAMYEIEN
VEGQGEDKDGLYEPSVWFNATWDAWTFSLAMYQEGTVEYS
SMTRGSYFDRPEFELRYRFIGTDDLTLGLTGGFRNYGYHF
KDEHGAKDGSANMQRYKIQPDWDVKLTDDWRFSGWLAMYQ
FANDLEKTGYADSRVETETGFTWTINNIFSAKINYYLERG
FNMDGSRNNGEFATQEIRAYLPVSMGQTTLTPYTRLGLDR
WSNWDWQDDPSREGHDFNRLGLLYAYDFNNGLSMTLEYAY
EWQNHDEGKNDRFHYAGVGVNYAF.

In some embodiments, loop 1 comprises amino acids 37 to 52, loop 2 comprises amino acids 74 to 88, loop 3 comprises amino acids 117 to 132, loop 4 comprises amino acids 161 to 173, loop 5 comprises amino acids 198 to 217, loop 6 comprises amino acids 235 to 259, and loop 7 comprises amino acids 281 to 298 of SEQ ID NO:6. Therefore, in some embodiments, the heterologous peptide is inserted, replaces, or a combination thereof, within loop 1, i.e. amino acids 37 to 52 of SEQ ID NO:6. Therefore, in some embodiments, the heterologous peptide is inserted, replaces, or a combination thereof, within loop 2, i.e. amino acids 74 to 88 of SEQ ID NO:6. Therefore, in some embodiments, the heterologous peptide is inserted, replaces, or a combination thereof, within loop 3, i.e. amino acids 117 to 132 of SEQ ID NO:6. Therefore, in some embodiments, the heterologous peptide is inserted, replaces, or a combination thereof, within loop 4, i.e. amino acids 161 to 173 of SEQ ID NO:6. Therefore, in some embodiments, the heterologous peptide is inserted, replaces, or a combination thereof, within loop 5, i.e. amino acids 198 to 217 of SEQ ID NO:6. Therefore, in some embodiments, the heterologous peptide is inserted, replaces, or a combination thereof, within loop 6, i.e. amino acids 235 to 259 of SEQ ID NO:6. Therefore, in some embodiments, the heterologous peptide is inserted, replaces, or a combination thereof, within loop 7, i.e. amino acids 281 to 298 of SEQ ID NO:6.

In some embodiments, OmpG has an amino acid sequence:

(SEQ ID NO: 7, A0A4Y8IIT9)
MSTLLKSAALVLCAGVSYAQASETTKQWEFNIGAMYEIEN
VEGQGDDKDGLYEPSVWFNATWDAWTLSLAMYQEGPVDYS
SMTRGTYFDRPEFELRYRFIGTDDFTFGLTGGFRNYGYHF
KDEHGAKDGSANMQRYKIQPDWDIKLTDDWRFGGWFAMYQ
FANDLEKTGYADSRVETETGFTWTINETFAAKVNYYLERG
FNMDRSRNNGEFSTQEIRAYLPISLGQTTLTPYTRLGLDR
WSNWDWQDDPEREGHDFNRLGLLYAYDFNNGLSMTLEYAY
EWENHDEGESDRFHYAGVGVNYAF.

In some embodiments, loop 1 comprises amino acids 37 to 52, loop 2 comprises amino acids 74 to 88, loop 3 comprises amino acids 117 to 132, loop 4 comprises amino acids 161 to 173, loop 5 comprises amino acids 198 to 217, loop 6 comprises amino acids 235 to 259, and loop 7 comprises amino acids 281 to 298 of SEQ ID NO:7. Therefore, in some embodiments, the heterologous peptide is inserted, replaces, or a combination thereof, within loop 1, i.e. amino acids 37 to 52 of SEQ ID NO:7. Therefore, in some embodiments, the heterologous peptide is inserted, replaces, or a combination thereof, within loop 2, i.e. amino acids 74 to 88 of SEQ ID NO:7. Therefore, in some embodiments, the heterologous peptide is inserted, replaces, or a combination thereof, within loop 3, i.e. amino acids 117 to 132 of SEQ ID NO:7. Therefore, in some embodiments, the heterologous peptide is inserted, replaces, or a combination thereof, within loop 4, i.e. amino acids 161 to 173 of SEQ ID NO:7. Therefore, in some embodiments, the heterologous peptide is inserted, replaces, or a combination thereof, within loop 5, i.e. amino acids 198 to 217 of SEQ ID NO:7. Therefore, in some embodiments, the heterologous peptide is inserted, replaces, or a combination thereof, within loop 6, i.e. amino acids 235 to 259 of SEQ ID NO:7. Therefore, in some embodiments, the heterologous peptide is inserted, replaces, or a combination thereof, within loop 7, i.e. amino acids 281 to 298 of SEQ ID NO:7.

In some embodiments, OmpG has an amino acid sequence:

(SEQ ID NO: 8, A0A381G793)
MKTLLSSSALLICAGMACAQAADNKDWHENIGAMYEIENV
EGYGEDMDGLAEPSVYFNASNGPWRISLAYYQEGPVDYSA
GKRGTWFDRPELEVHYQILESDDFSFGLTGGFRNYGYHYV
NEAGKDTANMQRWKVAPDWNVKLTDDLRFSGWLAMYQFVN
DLTTTGYSDSRVESETGLNYTFNETVGLTVNYYLERGFNL
AEHRNNGEFSTQEIRAYLPISLGNTTLTPYTRIGLDRWSN
WDWRDDPEREGHDFNRLGLQYAYDFQNGVSMTLEYAYEWE
DHDEGDSDRFHYAGIGVNYAF.

In some embodiments, loop 1 comprises amino acids 36 to 51, loop 2 comprises amino acids 73 to 87, loop 3 comprises amino acids 116 to 129, loop 4 comprises amino acids 158 to 170, loop 5 comprises amino acids 195 to 214, loop 6 comprises amino acids 232 to 256, and loop 7 comprises amino acids 278 to 295 of SEQ ID NO:8. Therefore, in some embodiments, the heterologous peptide is inserted, replaces, or a combination thereof, within loop 1, i.e. amino acids 36 to 51 of SEQ ID NO:8. Therefore, in some embodiments, the heterologous peptide is inserted, replaces, or a combination thereof, within loop 2, i.e. amino acids 73 to 87 of SEQ ID NO:8. Therefore, in some embodiments, the heterologous peptide is inserted, replaces, or a combination thereof, within loop 3, i.e. amino acids 116 to 129 of SEQ ID NO:8. Therefore, in some embodiments, the heterologous peptide is inserted, replaces, or a combination thereof, within loop 4, i.e. amino acids 158 to 170 of SEQ ID NO:8. Therefore, in some embodiments, the heterologous peptide is inserted, replaces, or a combination thereof, within loop 5, i.e. amino acids 195 to 214 of SEQ ID NO:8. Therefore, in some embodiments, the heterologous peptide is inserted, replaces, or a combination thereof, within loop 6, i.e. amino acids 232 to 256 of SEQ ID NO:8. Therefore, in some embodiments, the heterologous peptide is inserted, replaces, or a combination thereof, within loop 7, i.e. amino acids 278 to 295 of SEQ ID NO:8.

In some embodiments, OmpG has an amino acid sequence:

(SEQ ID NO: 9, A0A8B2FLU0)
MKTLLSSTALLMCAGMACAQAAENNDWHFNVGAMYEIENV
EGQGEDMDGLAEPSVYFNAANGPWKISLAYYQEGPVDYSA
GKRGTWFDRPELEVRYQFLESDDVNFGLTGGFRNYGYHYV
NEPGKDTANMQRWKVSPDWDVKISDNVRFGGWLSLYQFVN
DLSTTGYSDSRVETETGFTWNINETFSLVTNYYLERGFNI
DKSRNNGEFSTQEIRAYLPVALGNTTLTPYTRIGLDRWSN
WDWQDDIEREGHDFNRLGMLYAYDFQNGLSMTLEYAFEWQ
DHDEGERDHFHYAGVGVNYAF.

In some embodiments, loop 1 comprises amino acids 36 to 51, loop 2 comprises amino acids 73 to 87, loop 3 comprises amino acids 116 to 129, loop 4 comprises amino acids 158 to 170, loop 5 comprises amino acids 195 to 214, loop 6 comprises amino acids 232 to 256, and loop 7 comprises amino acids 278 to 295 of SEQ ID NO:9. Therefore, in some embodiments, the heterologous peptide is inserted, replaces, or a combination thereof, within loop 1, i.e. amino acids 36 to 51 of SEQ ID NO:9. Therefore, in some embodiments, the heterologous peptide is inserted, replaces, or a combination thereof, within loop 2, i.e. amino acids 73 to 87 of SEQ ID NO:9. Therefore, in some embodiments, the heterologous peptide is inserted, replaces, or a combination thereof, within loop 3, i.e. amino acids 116 to 129 of SEQ ID NO:9. Therefore, in some embodiments, the heterologous peptide is inserted, replaces, or a combination thereof, within loop 4, i.e. amino acids 158 to 170 of SEQ ID NO:9. Therefore, in some embodiments, the heterologous peptide is inserted, replaces, or a combination thereof, within loop 5, i.e. amino acids 195 to 214 of SEQ ID NO:9. Therefore, in some embodiments, the heterologous peptide is inserted, replaces, or a combination thereof, within loop 6, i.e. amino acids 232 to 256 of SEQ ID NO:9. Therefore, in some embodiments, the heterologous peptide is inserted, replaces, or a combination thereof, within loop 7, i.e. amino acids 278 to 295 of SEQ ID NO:9.

In some embodiments, the nanopore comprise a single heterologous peptide inserted within loop 1, 2, 3, 4, 5, 6, or 7. In some embodiments, the nanopore comprise a 2, 3, 4, 5, 6, or 7 different heterologous peptides independently inserted within loop 1, 2, 3, 4, 5, 6, and/or 7, wherein each loop has a unique gating signature for multiplex detection.

In some embodiments, the heterologous peptide is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, or 100 amino acids in length. In some embodiments, the length of the peptide matches the number of amino acids replaced in the loop to maintain the loop lengths. In other embodiments, the length of the loop increases with the insertion of the heterologous peptide, for example, in some embodiments, the loop increases by 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 70, 80, 90, or 100 amino acids.

In some embodiments, the heterologous peptide is an epitope and the nanopore is used to detect or screen for an antibody that binds the epitope. For example, in some embodiments, the nanopore is used to distinguish between hybridomas. Tau protein sequences are described, for example, in Horowitz, P M, et al. J. Neuroscience 2004, 24(36):7895-790, which is incorporated by reference in its entirety for these sequences and uses to screen hybridomas.

SH2 domain proteins sequences are described, for example, in Colwill, K, et al. Nature Methods 2011 8:551-558, which is incorporated by reference in its entirety for these sequences and uses to screen hybridomas.

Transitin sequences (e.g. LREEHRDLQE (SEQ ID NO:10) are described, for example, in Darenfed, H, et al. Histochemistry and Cell Biology 2001 116:397-409, which is incorporated by reference in its entirety for these sequences and uses to screen hybridomas.

Drosophila nuclear lamin sequences (e.g. RPPSAGP (SEQ ID NO: 11) are described, for example, in J Cell Sci 1995 108(9):3137-3144, which is incorporated by reference in its entirety for these sequences and uses to screen hybridomas.

In some embodiments, the peptide is a cAMP-specific phosphodieserase PDE4A8 epitope: LKPPPQHLWRQPRTPIRIQQ (SEQ ID NO:12).

In some embodiments, the peptide is a human c-MYC peptide: EQKLISEEDL (SEQ ID NO:13).

In some embodiments, the nanopore use used to screen a sample for antibodies that bind a disease biomarker. In other embodiments, the heterologous peptide is a peptide aptamer or nanobody that binds a disease biomarker, in this instance, a structure like a surface-exposed protein on a pathogen for detection. Further, the disclosed nanopore can also be used to subtype a pathogen in addition to detection, such as virus. For example, human noroviruses are extremely prevalent in the population and also quite diverse, making both rapid, portable detection and subtyping crucial to identifying outbreaks and/or attributing sources of transmission or contamination. Numerous peptides that bind the outer capsid protein of noroviruses have been reported that have been used for detection (Rogers, J. D., et al. J. Clin. Microbiol. 2013, 51(6):1803-1808; Baek, S. H., et al. Biosens. Bioelectron. 2018, No. June, 0-1; Hwang, H. J., et al. Biosens. Bioelectron. 2017, 87:164-170; Hurwitz, A. M., et al. Protein Eng. Des. Sel. 2016, 1-11; Tan, M., et al. Virology 2008, 382 (1), 115-123; Tan, M., et al. J. Virol. 2005, 79(22):14017-14030). This concept could be applied for nearly all additional pathogen targets so long as there is a peptide sequence capable of binding, including coronaviruses (Yang, F, et al. ACS Omega 2022, 7(4):3203-3211; Ren, X, et al. Virology 410(2):299-306; Liu, Z-X, et al. Biochem. Biophys. Res. Comm. 2005, 329(2):437-444), bacterial pathogens like Salmonella enterica (Agrawal, S, et al. J Biotechnol. 2016 Aug. 10; 231:40-45; Steingroewer, J, et al. J Magnetism and Magnetic Materials. 311(1):295-299; Morton, J, et al. J Appl Microbiol. 2013 115(1):271-81), and other food contaminants and toxins like mycotoxins (aflatoxin, ochratoxin, etc.) (Hou, S-L, et al. Talanta. 2019 Mar. 1; 194:919-924; Sun, W, et al. Molecules. 2021 Dec. 17; 26(24):7652; Rangnoi, K, et al. Mol Biotechnol. 2011 49(3):240-9).

In some embodiments, binding of a target to the heterologous peptide results in a conformational change in the nanopore that alters ion current flow through the pore. In some embodiments, the change in ion current flow is a result of a change in gating due to the conformational change. In some embodiments, the nanopore detects target binding in a manner that does not involve entry of the target into the pore lumen.

In certain embodiments, determining the gating pattern comprises performing a single channel recording of ion flow through the nanopore. In some embodiments, the single channel recording is performed while exposing the nanopore to a potential in a range of −250 to +250 mV or −100 to +100 mV. In certain embodiments, the single channel recording is performed while exposing the nanopore to a potential in a range of −50 mV to +50 mV. In some embodiments, binding of the target to the heterologous peptide is detected based on an alteration (e.g., an increase or a reduction) in the gating pattern resulting from exposing the nanopore to a target molecule. In some embodiments, binding of the target to the heterologous peptide is detected based on an alteration (e.g., an increase or a reduction) in amplitude and/or frequency of gating. In some embodiments, alterations in gating patterns are due to the interaction of bound analyte with one or more loops at the ligand side of a beta barrel of a nanopore. In some embodiments, an analyte can slow down or otherwise alter the movement of a loop, e.g., by containing or tethering the loop, or altering the loop such that it is stuck in a “half-open” or “closed” position, etc.

In certain embodiments, the reduction is a reduction of the frequency and/or amplitude of gating events. In some embodiments, determining the gating pattern comprises determining a gating frequency (f, events/s). In certain embodiments, the gating frequency is a relationship between a total number of gating events and a recording time. In some embodiments, determining the gating pattern comprises determining a gating probability (Pgating). In certain embodiments, the gating probability is a relationship between a total time for which a pore is in a closed or partially closed state and a total recording time (total open and closed time). In some embodiments, determining the gating pattern comprises determining loop dynamics to detect ligand-target interactions. In some embodiments, a gating pattern change is from “quiet” to “noisy”. However, in some embodiments, a gating pattern change is from noisy to quiet. In some embodiments, a reduction is gating pattern from noisy to quiet occurs where a reduction in the ionic current noise is greater than 10%.

In certain embodiments, the membrane preparation is a synthetic membrane preparation. In some embodiments, the membrane preparation is a planar lipid bilayer. In certain embodiments, the membrane preparation is a micelle. In some embodiments, the membrane preparation is a membrane of a biological cell. In certain embodiments, the biological cell is a bacterium. In some embodiments, the biological cell is a eukaryotic cell.

Disclosed herein is a composition comprising one or more nanopores disposed in a membrane preparation. For example, the nanopore can have a plurality of β-strands connected by a plurality of flexible loops on a first side of the membrane preparation and a plurality of short turns on a second side of the membrane preparation, in which at least one of the flexible loops comprises a heterologous peptide that binds a target molecule.

In some embodiments, a plurality of unique nanopore disposed in the membrane preparation, each of the plurality of nanopores having a unique heterologous peptide. Also disclosed is a composition comprising a plurality of membrane preparations, each membrane preparation having a unique nanopore disposed in the membrane preparation, each of the nanopores having a unique heterologous peptide.

In some embodiments, the nanopores are disposed in a membrane preparation such that on one side of the membrane preparation the nanopores are exposed to a common chamber and on the other side of the membrane preparation each nanopores is exposed to a separate chamber. In some embodiments, the extracellular loop of each nanopores is exposed to the common chamber. In some embodiments, the extracellular loop of each nanopores is exposed to the separate chamber. In some embodiments, the nanopores are configured for multi-nanopore high-throughput measurements. In some embodiments, a set of nanopores are provided in a membrane preparation (e.g., in a 2-dimensional array) such that at one side of the membrane preparation the multiple nanopores are exposed to a single chamber, and at the other side of the membrane preparation, each nanopore is exposed to an individual chamber.

In some embodiments, a nucleic acid is provided that encodes an engineered nanopores disclosed herein.

Disclosed herein is a method for detecting a target that involves exposing a nanopore disclosed herein to the target, wherein the nanopore is disposed in a membrane preparation, and wherein the nanopore comprises a heterologous peptide in one or more of the flexible loops that binds the target; assessing ion current flow through the nanopore; and detecting binding of the heterologous peptide to the target based on the ion current flow. In some embodiments, ligand-target binding is indicated by a block in ion current flow.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: Single-Molecule Multiplex Protein Detection Using an Engineered OmpG Nanopore

Results and Discussion

Development of Flow Cytometry Assay to Assess OmpG Motif Display

Attachment of affinity reagents via chemical labeling can result in a population heterogeneity, potentially confounding later analysis, whereas genetic modification results in atomically precise constructs exhibiting the desired properties. Therefore, when developing a multiplex sensor, efforts were made to utilize genetically encoded peptide motifs. Prior work utilizing biotin-conjugated OmpG constructs observed that biosensor sensitivity is impacted by the attachment point of the affinity reagent, i.e., the display loop, which may also be a factor with loop-integrated peptide motifs. However, the predominant analytical technique of single-channel current recording has limited throughput when characterizing novel construct/analyte interactions, thus a more rapid screening assay was desirable to initially probe the viability of an affinity reagent display site from an ensemble measurement before moving to single-channel analysis.

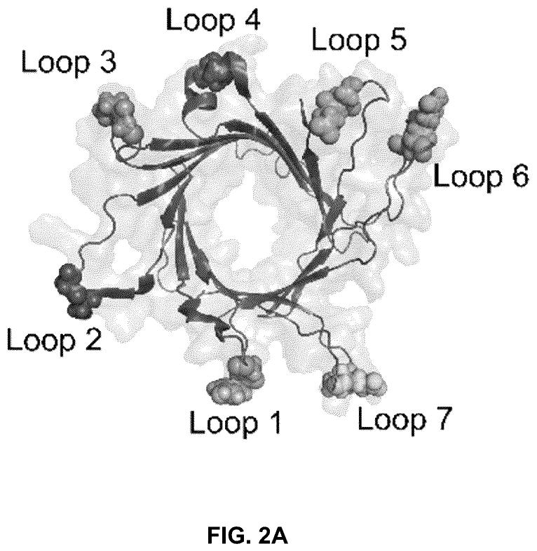

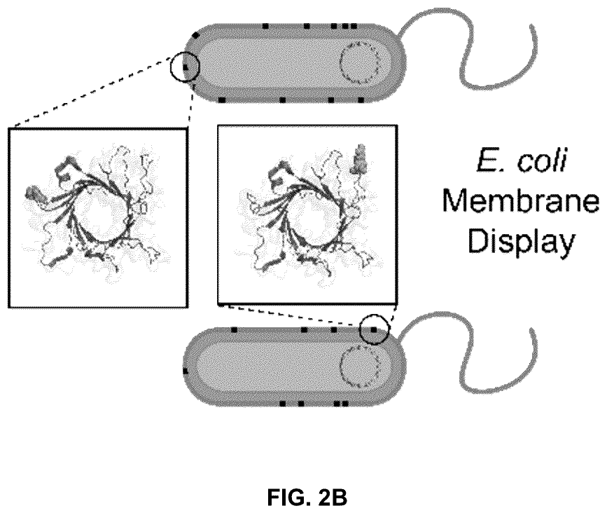

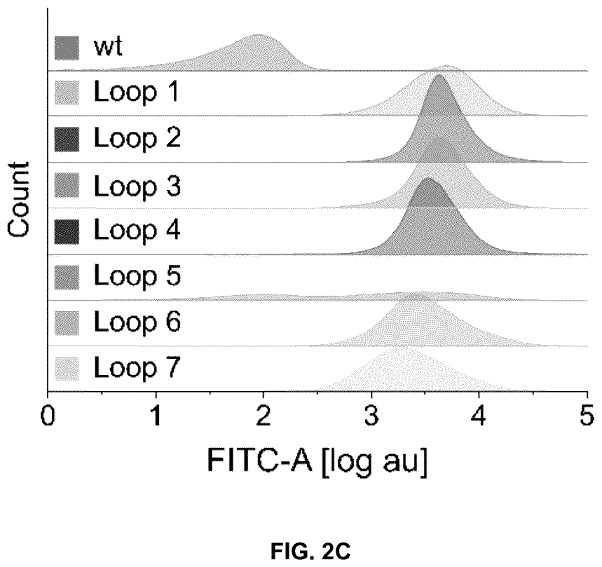

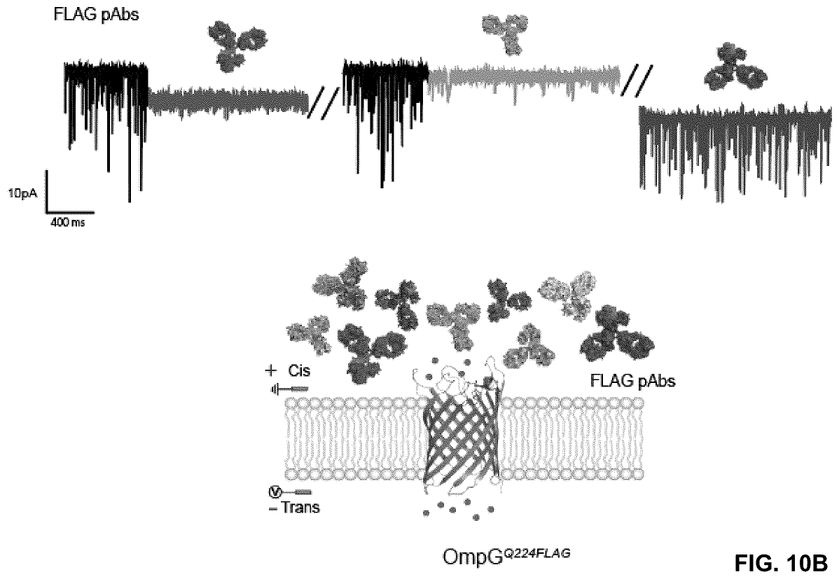

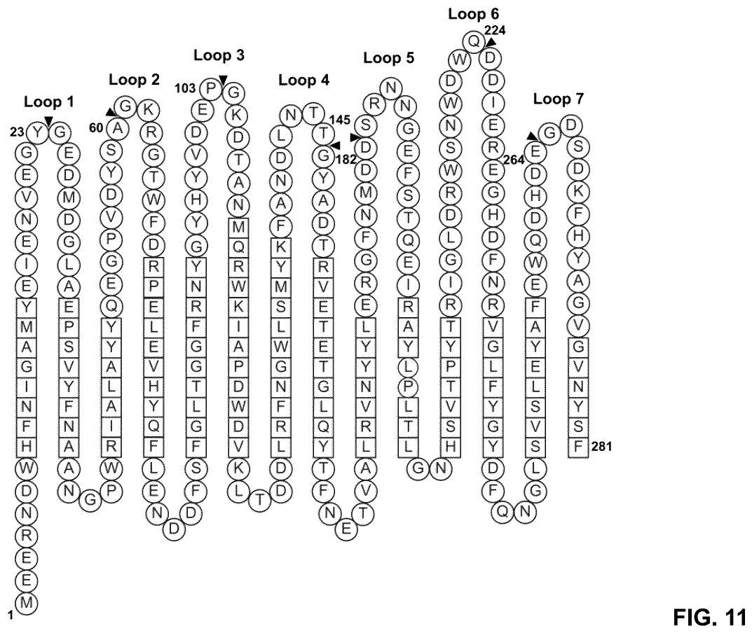

Flow cytometry was well suited for this task as OmpG is an endogenous Escherichia coli (E. coli) outer membrane porin allowing for efficient display of engineered peptides sequence(s) on the cell surface where they are accessible to protein analyte(s). Constructs displaying the FLAG motif (GDYKDDDDKG, SEQ ID NO:14) in the center of each of OmpG's seven loops (FIG. 2A) were generated using oligos delineated in Table 1 (further detail regarding insertion site shown in FIG. 11) and termed OmpG^{Ln-FLAG}, with n indicating the display loop number. The FLAG motif was selected for this assay due to the availability of monoclonal tag-specific antibodies and prior use in OMP display.

TABLE 1
List of oligonucleotides used
to generate OmpG constructs.
Underlined sequence was inserted
into the indicated loop.

| Display | |
|---|---|
| Loop/Motif | Oligonucleotide Sequence |
| Loop 1/FLAG | Forward: |
| | 5′-AAAGATGACGATGATAAAGGAGGCG |
| | AAGATATGGATGGGCTGG-3′ |
| | (SEQ ID NO: 15) |
| | Reverse: |
| | 5′-ATCATCGTCATCTTTATAATCACCA |
| | TAACCCTCGACGTTTTCTATTTCG-3′ |
| | (SEQ ID NO: 16) |
| Loop 2/FLAG | Forward: |
| | 5′-TATAAAGATGACGATGATAAAGGTG |
| | GTAAACGTGGAACG-3′ |
| | (SEQ ID NO: 17) |
| | Reverse: |
| | 5′-ATCGTCATCTTTATAATCGCCCGCG |
| | CTATAATCTAC-3′ |
| | (SEQ ID NO: 18) |
| Loop 3/FLAG | Forward: |
| | 5′-TATAAAGATGACGATGATAAAGGTG |
| | GTAAAGACACGGCG-3′ |
| | (SEQ ID NO: 19) |
| | Reverse: |
| | 5′-ATCGTCATCTTTATAATCGCCCGGT |
| | TCATCAACGTAG-3′ |
| | (SEQ ID NO: 20) |
| Loop 4 FLAG | Forward: |
| | 5′-AAAGATGACGATGATAAAGGAGGTT |
| | ACGCTGATACCCGTGTCG-3′ |
| | (SEQ ID NO: 21) |
| | Reverse: |
| | 5′-ATCATCGTCATCTTTATAATCACCG |
| | GTAGTGTTCAGATCGTTGGC-3′ |
| | (SEQ ID NO: 22) |
| Loop 5 FLAG | Forward: |
| | 5′-TAAAGATGACGATGATAAAGGAAGC |
| | CGCAATAACGGTGAGTTTTCC-3′ |
| | (SEQ ID NO: 23) |
| | Reverse: |
| | 5′-TCATCGTCATCTTTATAATCACCGT |
| | CGTCCATATTGAAGCCGCGC-3′ |
| | (SEQ ID NO: 24) |
| Loop 6 FLAG | Forward: |
| | 5′-TATAAAGATGACGATGATAAAGGAG |
| | ATGATATTGAACGTGAAG-3′ |
| | (SEQ ID NO: 25) |
| | Reverse: |
| | 5′-TTTATCATCGTCATCTTTATAATCA |
| | CCCTGCCAGTCCCAGTTACTCC-3′ |
| | (SEQ ID NO: 26) |
| Loop 7 FLAG | Forward: |
| | 5′-TATAAAGATGACGATGATAAAGGAG |
| | GCGACAGTGATAAATTCCATTATGC |
| | -3′ |
| | (SEQ ID NO: 27) |
| | Reverse: |
| | 5′-ATCATCGTCATCTTTATAATCACCT |
| | TCGTCGTGATCCTGCCACTC-3′ |
| | (SEQ ID NO: 28) |
| Loop 6 | Forward: |
| | 5′-GAAGCGTGGCTGGGCGCGCGTGGTG |
| | ATATTGAACGTG-3′ |
| | (SEQ ID NO: 29) |
| Nanotag | Reverse: |
| | 5′-GCCCAGCCACGCTTCCACATCGCCC |
| | TGCCAGTCCCAG-3′ |
| | (SEQ ID NO: 30) |
| Loop 6 SA | Forward: |
| | 5′-GTATGAACGTGTGTGATGATATTGA |
| | ACGTGAAGGCC-3′ |
| | (SEQ ID NO: 31) |
| Consensus | Reverse: |
| | 5′-CACGTTCATACAGATCTGCCAGTCC |
| | CAGTTACTCCAGC-3′ |
| | (SEQ ID NO: 32) |
| Loop 6 SA-3 | Forward: |
| | 5′- |
| | TGTATGAACATCTGTTGGACTGGAG |
| | AAACGCAGGATGATATTGAACGTGA |
| | AGGCCATGATTTTAACCG-3′ |
| | (SEQ ID NO: 33) |
| | Reverse: |
| | 5′- |
| | GTCCAACAGATGTTCATACATATGA |
| | GCACAGTCTGCCAGTCCCAGTTACT |
| | CCAGCGATCCAGCCC-3′ |
| | (SEQ ID NO: 34) |
| Loop 6 SA-3 | Forward: |
| Truncate | 5′-TGTATGAACATTTGTTGGACCGATG |
| | ATATTGAACGTGAAGGCCATG-3′ |
| | (SEQ ID NO: 35) |
| Loop 6 SA-3.1 | Forward: |
| | 5′-CATTTGTATGAACATTTGTTGGACC |
| | GGAGATGATATTGAACGTGAAGGCC |
| | ATG-3′ |
| | (SEQ ID NO: 36) |
| | Reverse: |
| | 5′-CCAACAAATGTTCATACAAATGAGC |
| | ACACCCTGCCAGTCCCAGTTACTCC |
| | AGC-3′ |
| | (SEQ ID NO: 37) |
| Loop 6 SA-1 | Forward: |
| Truncate | 5′-TTTGTCAGAATGTGTGTTATTACGA |
| | TGATATTGAACGTGAAGGCC-3′ |
| | (SEQ ID NO: 38) |
| | Reverse: |
| | 5′-ACACACATTCTGACAAATTTCGAGC |
| | TGCCAGTCCCAGTTACTCCAGC-3′ |
| | (SEQ ID NO: 39) |
| Loop 6 SA1.1 | Forward: |
| | 5′ -ATTTGTCAGAATGTGTGTTATTACG |
| | GAGATGATATTGAACGTGAAGGCCATG-3′ |
| | (SEQ ID NO: 40) |
| | Reverse: |
| | 5′-ACACACATTCTGACAAATTTCGAGA |
| | CCCTGCCAGTCCCAGTTACTCCAGC-3′ |
| | (SEQ ID NO: 41) |
| Loop 3 SA1.1 | Forward: |
| | 5′-ATTTGTCAGAATGTGTGTTATTACG |
| | GAGGTAAAGACACGGCGAATATGC-3′ |
| | (SEQ ID NO: 42) |
| | Reverse: |
| | 5′-ACACACATTCTGACAAATTTCGAGA |
| | CCCGGTTCATCAACGTAGTGATAAC-3′ |
| | (SEQ ID NO: 43) |
| SsdeI | Forward: |
| | 5′-GAGGAAAGGAACGACTGGCACTTTA |
| | ATATCGG-3′ |
| | (SEQ ID NO: 44) |
| | Reverse: |
| | 5′-CATATGTATATCTCCTTCTTAAAGT |
| | TAAACAAAATTATTTCTAGAGG-3′ |
| | (SEQ ID NO: 45) |

Seven OmpG^{Ln-FLAG} constructs were separately expressed at the outer membrane of BL21 (DE3) E. coli (FIG. 2B). Display of the FLAG motif was then assessed by flow cytometry using a sandwich-style labeling via monoclonal mouse anti-FLAG antibody FG4R and goat anti-mouse polyclonal antibody conjugated to fluorescein (FITC). There was a clear increase FITC channel emission relative to OmpG^{wt} for most constructs, as indicated by a rightward shift in counts per average FITC-A. Loops 1/6/7 exhibited a wider distribution in fluorescence intensity, whereas loops 2/3/4 exhibited tighter clustering of their fluorescence signal, suggesting differences in labeling efficiency as a function of motif location. Interestingly, OmpG^{L5-FLAG} showed almost no FITC channel emission with average counts below OmpG^{wt}, suggesting its membrane display resulted in significant negative fitness for the expressing population however the underlying cause for this observation is not currently understood. Overall, this assay was an effective means to rapidly assess display efficiency, with clear trends discernible even from qualitative comparison. Constructs displaying the FLAG motif in loops 2, 3, 4, and 6 were selected for further single-channel analysis due to their strong fluorescent signal in this assay and the utility of these loops for analyte detection in our prior study.

Monoclonal Anti-FLAG Antibody FG4R Detection Via Loop 3 Peptide Display

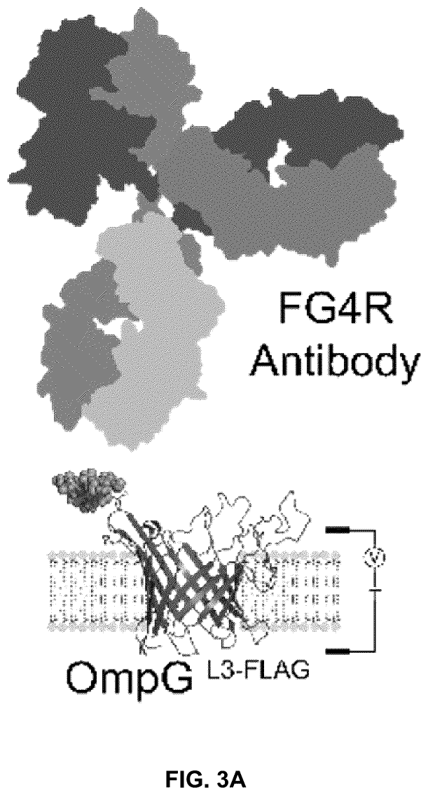

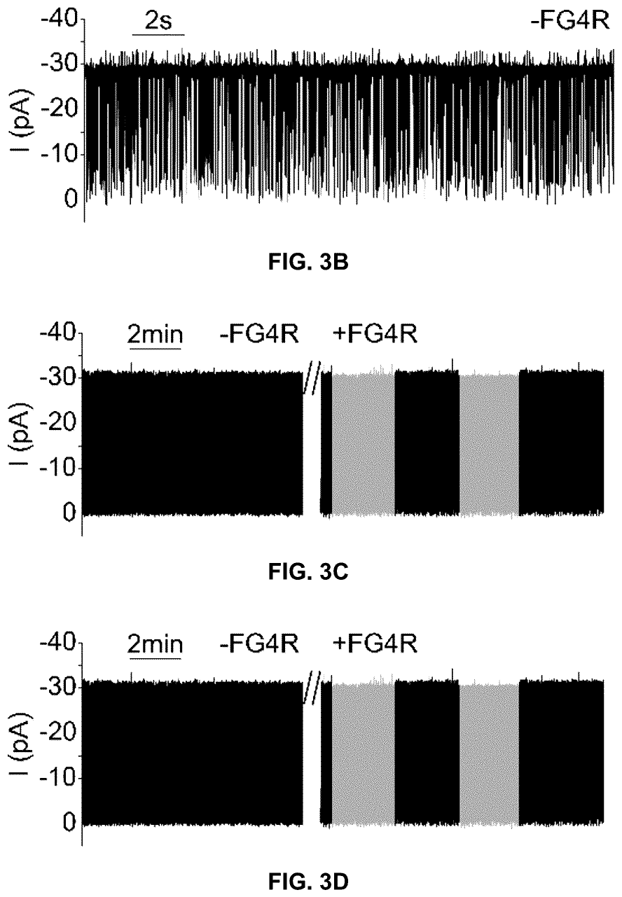

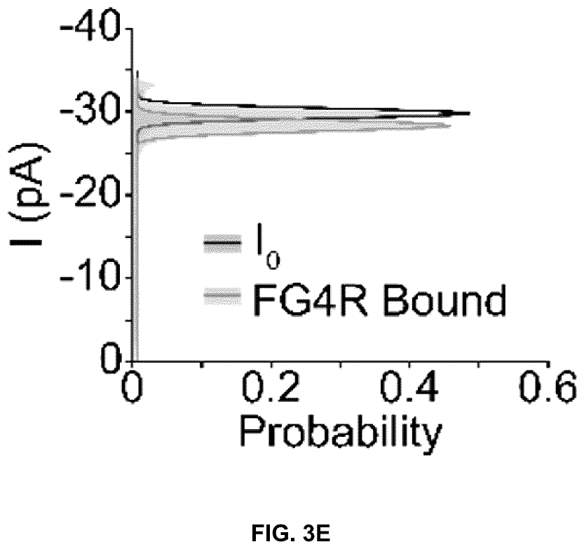

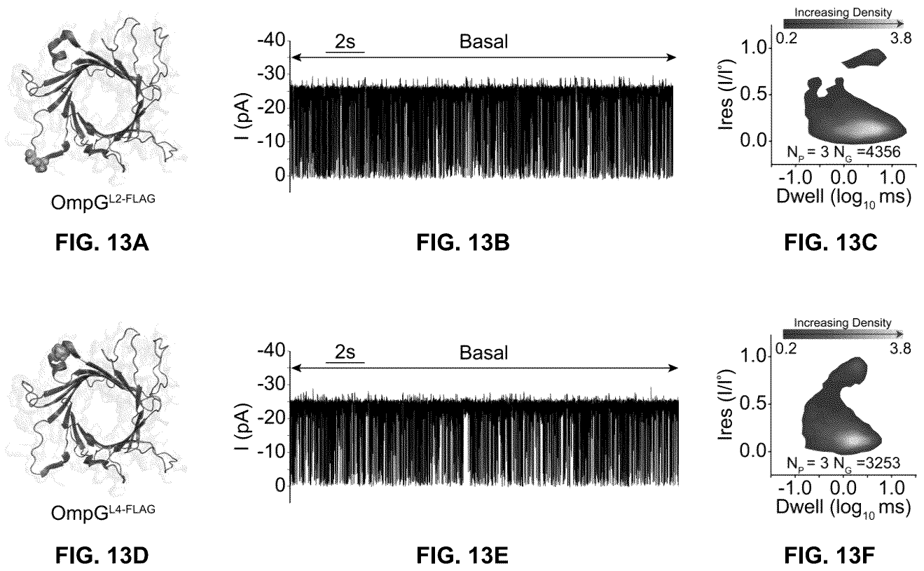

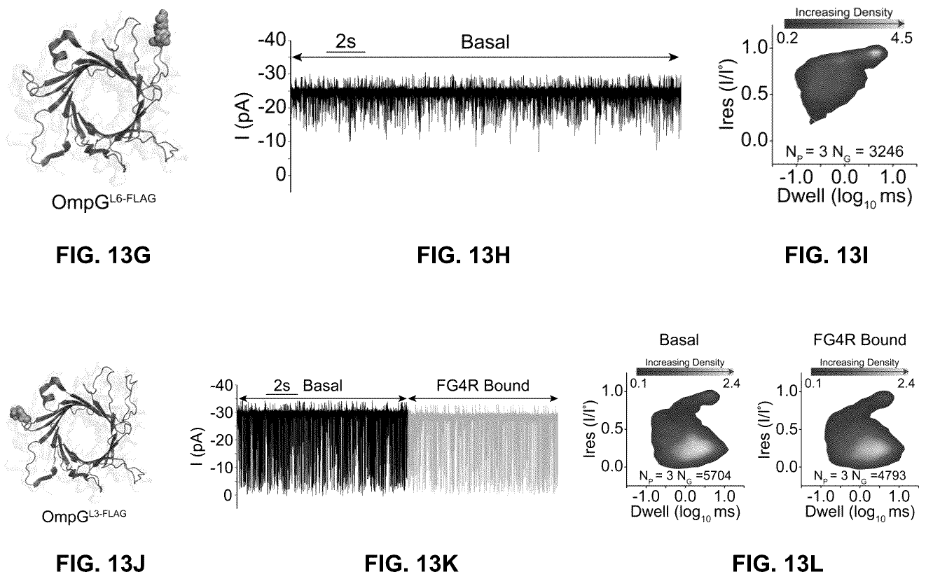

Single-channel current measurement of OmpG^{L2-FLAG}, OmpG^{L3-FLAG}, and OmpG^{L4-FLAG} basal gating strongly resembled OmpG^{wt}, as characterized by rapid fluctuations between high and low conductance states, indicating hosting the FLAG motif at these positions did not meaningfully alter gating dynamics (FIGS. 3B, 13B, 13E). In contrast, OmpG^{L6-FLAG} exhibited a significant reduction in gating depth and duration (FIG. 13G), with similar behavior to a prior report of an OmpG construct with a pinned loop 6⁴¹. Addition of FG4R elicited no change for OmpG^{wt}, OmpG^{L2-FLAG}, OmpG^{L4-FLAG} and OmpG^{L6-FLAG} sensors (FIGS. 13B, 13E, 13H, & 14A) but resulted in a minute(s) long 5.02±1.11% (N=3, n=9, where N is the number of independent pores and n the number of individual binding events analyzed) reduction in pore conductance for OmpG^{L3-FLAG} (FIG. 3C-E). MOSAIC was used to further analyze trace segments covering the basal (I₀) and FG4R-bound states. This analysis showed a marginal, but not usefully discriminatory, reduction in gating frequency during OmpG^{L3-FLAG}/FG4R binding (FIG. 13I) leaving the reduction in current as the most effective parameter for event identification.

From this study antibody detection was observed at nanomolar concentration within tens of minutes via direct interaction of the analyte with a loop-displayed peptide motif. However, due to the inordinate recording time necessary to efficiently sample the minutes long FG4R binding events the exact binding kinetics were not determined. This highlights a potential limitation of this detection strategy; analytes with higher affinity generally exhibit greater pore residence precluding additional the detection of additional molecules, meaning binary detection but not quantitation can be effectively performed. Interestingly, while the majority of loops demonstrated effective interaction with FG4R from ensemble measurements only OmpG^{L3-FLAG} allowed for resolution of a discernible single-molecule binding signal. This observation suggests ensemble measurements are not sufficient to predict single-molecule binding behavior, and as such additional optimization of analyte binding pose along with pore gating behavior may be required to resolve analyte binding. Despite these limitations this strategy is potentially generalizable to other antibodies with known peptide antigens.

Streptavidin Detection Via Loop 6 Peptide Display

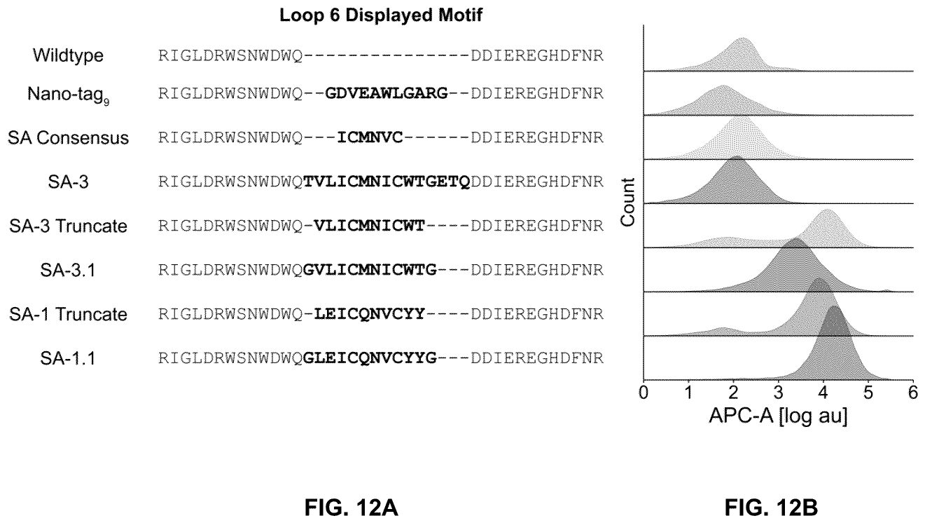

Two streptavidin-binding motifs were identified, Nano-tag₉ (DVEAWLGAR, SEQ ID NO:46) derived from a ribosome-display library, and SA-3 (TVLICMNICWTGETQ, SEQ ID NO:47) derived from an OmpA-scaffold bacterial display library with reported K_{d} of 17±4 nM and 4 nM respectively as potential candidates for sensor development. Additionally, truncations of SA-3 and SA-1 as well as the streptavidin-binding consensus sequence reported from Bessette et al. were considered (FIG. 12A) as there was concern increasing length into the display loop would hinder signal recognition. Utilizing the previously described bacterial display flow cytometry assay OmpG constructs harboring the mentioned streptavidin-binding motifs within OmpG loop6 via direct interaction with Alexa-647 conjugated streptavidin (referred to as Streptavidin-Alexa647) were screened (FIG. 12B). From this screen motif SA1.1 (LEICQNVCYY, SEQ ID NO:48) was identified, a truncation of 15 amino acid SA-1 around the reported OmpA-scaffold bacterial display library consensus, as the best candidate for single-molecule assessment based on Alexa-647 fluorescence emission in the APC channel (FIG. 12B). The fluorescence profiles observed in this screen did not correlate with the reported motif K_{d}, IE motifs with lower reported K_{d} did not necessarily exhibit higher fluorescence intensity or counts, showing further considerations of the local motif environment must be made in sensor design, highlighting the utility of the moderate throughput flow cytometry screen.

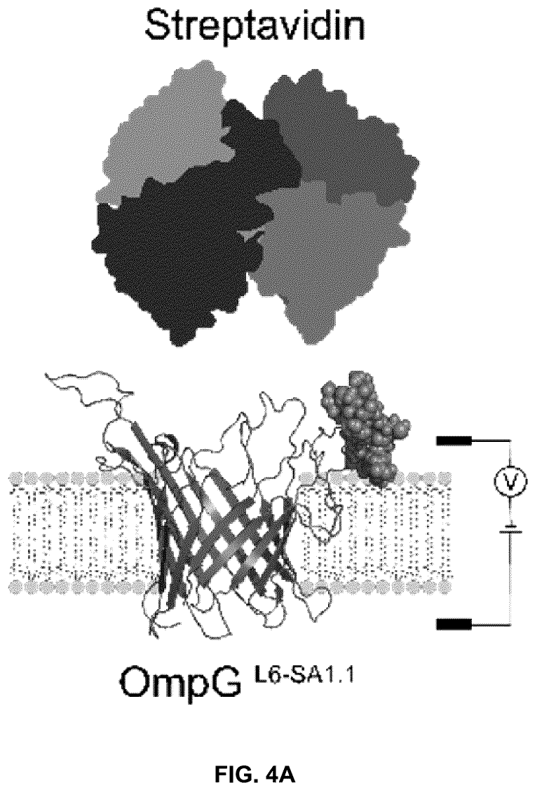

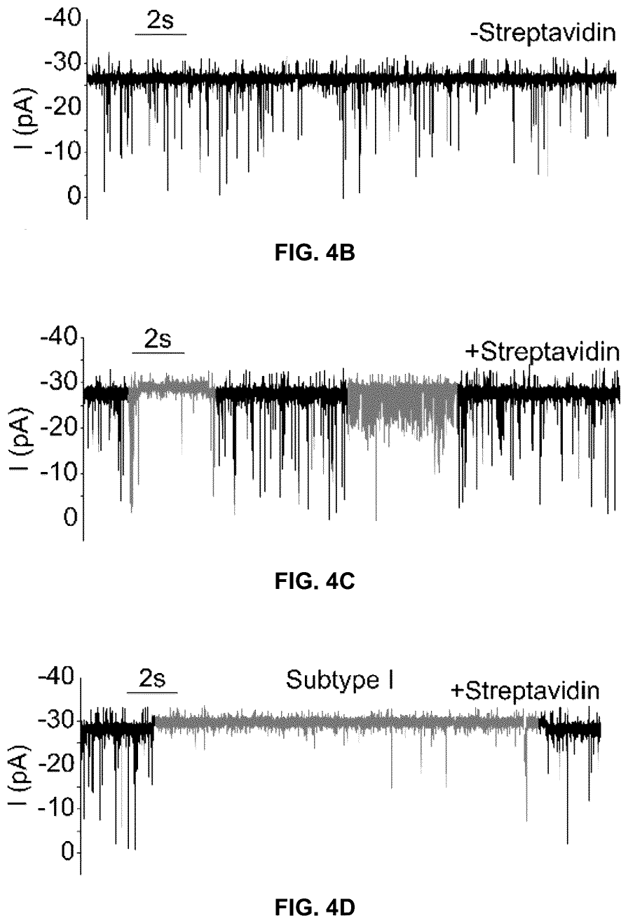

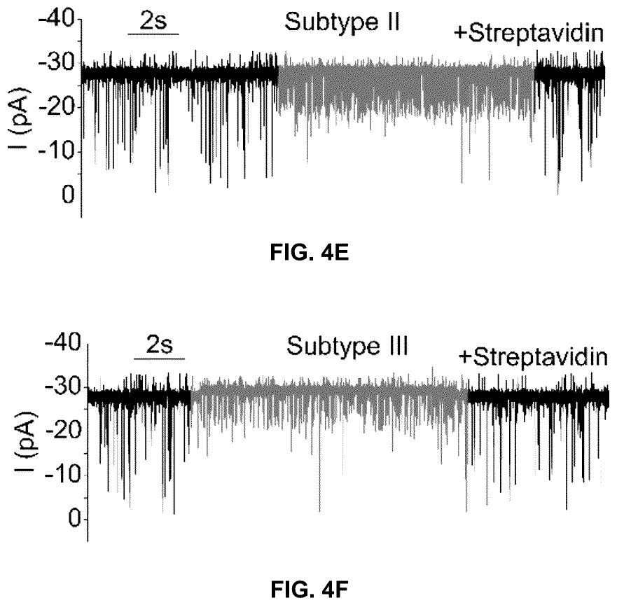

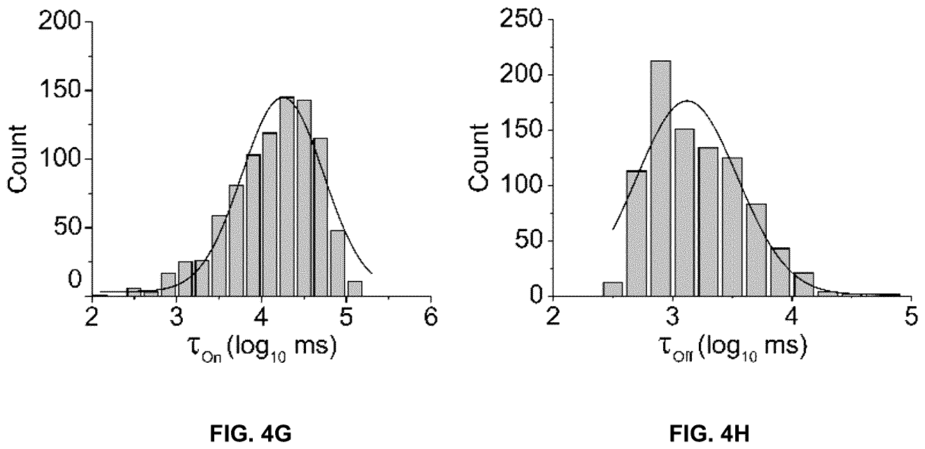

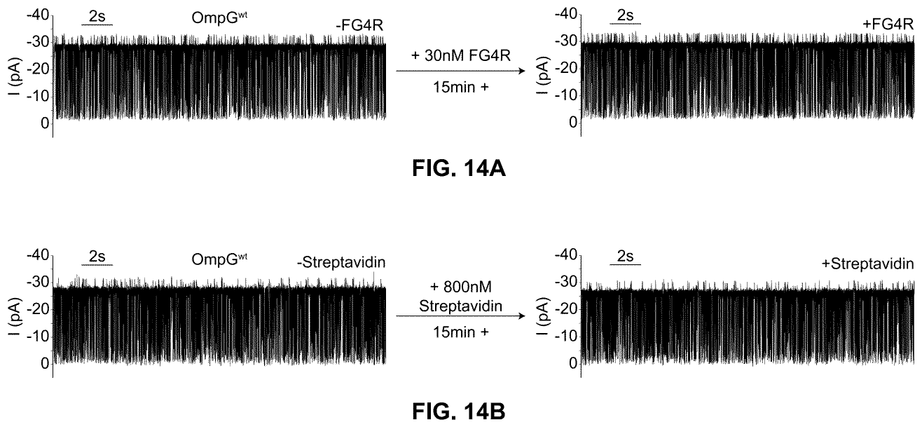

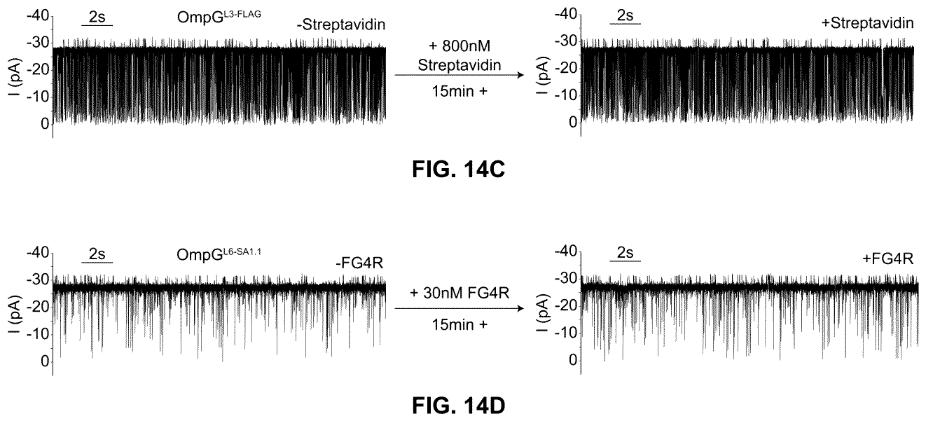

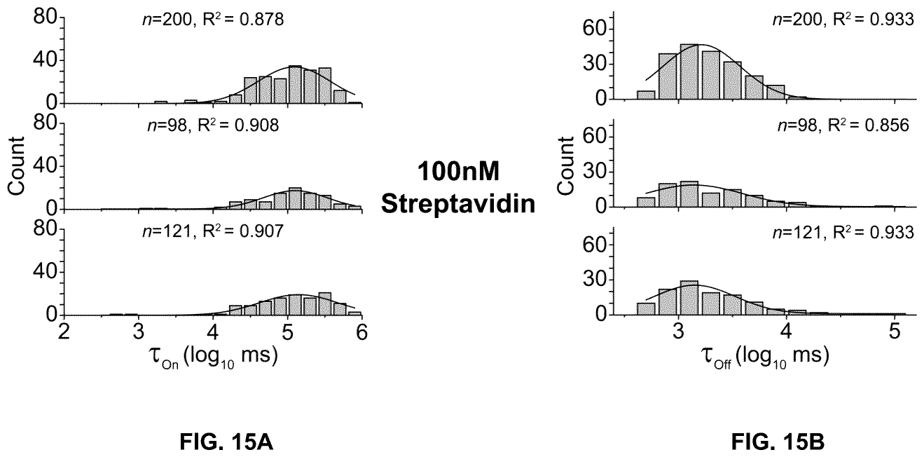

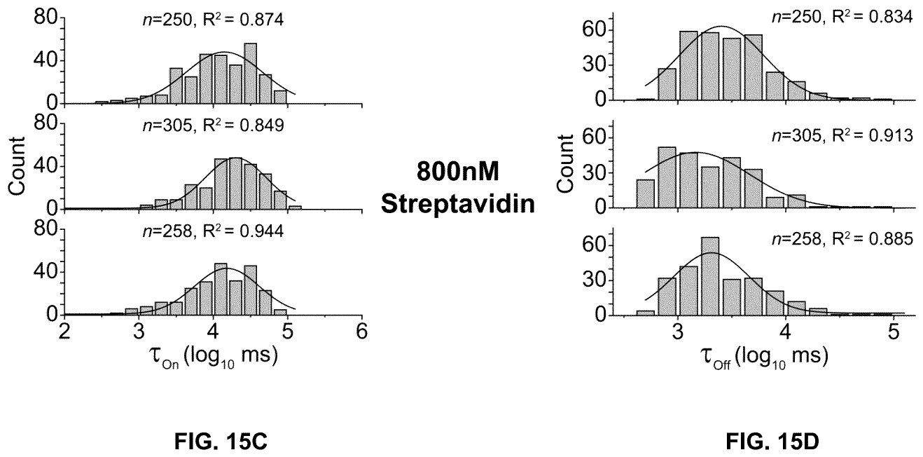

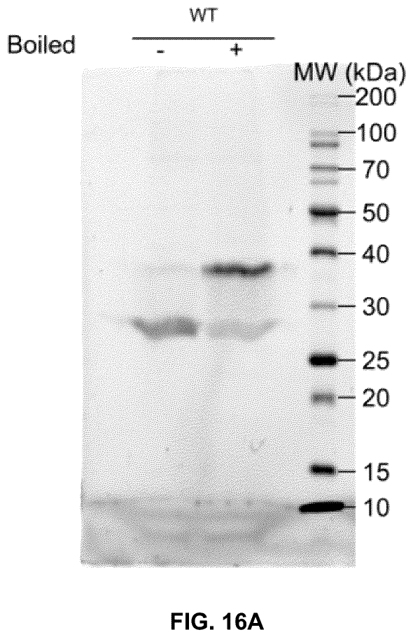

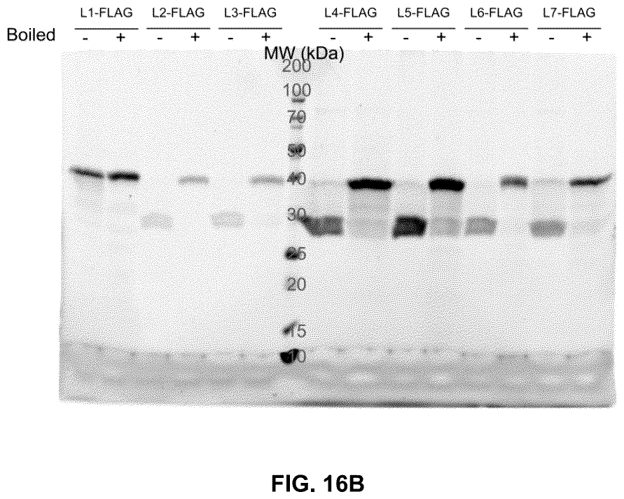

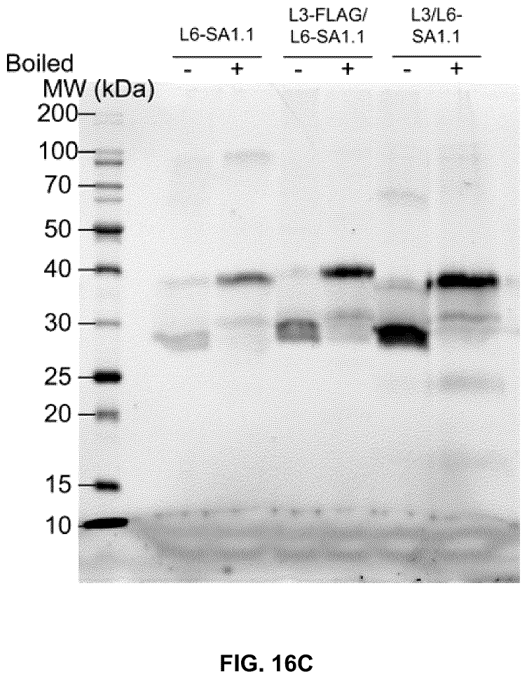

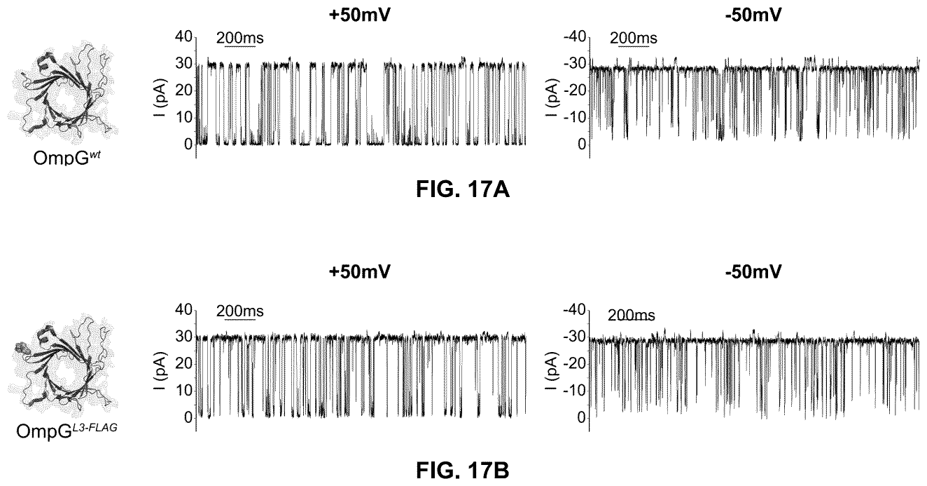

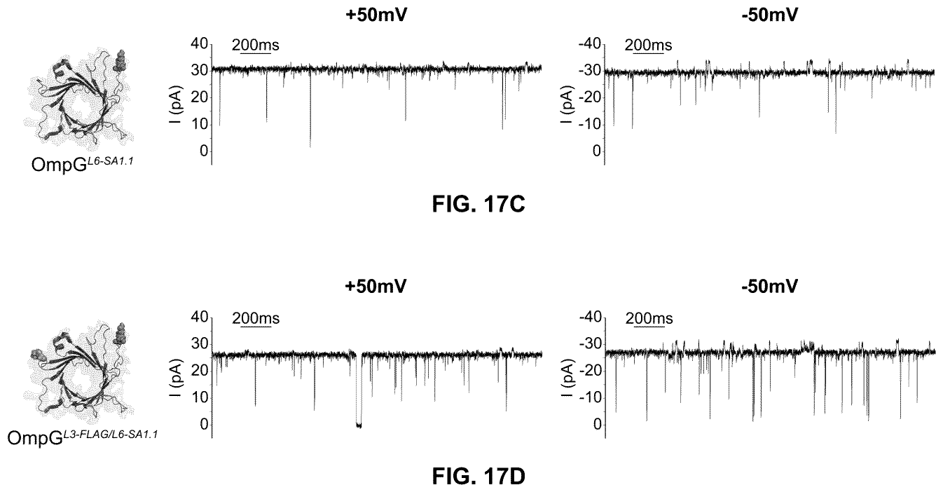

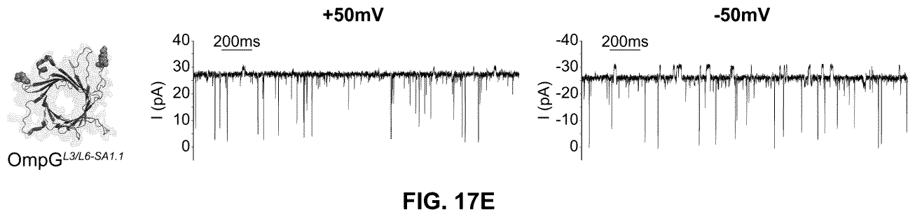

Single-channel current measurements of OmpG^{L6-SA1.1} revealed ‘quiet’ pore behavior, similar to OmpG^{L6-FLAG}, with an increased prevalence of ‘spikes’ to ˜50% pore conductance and an overall reduction in gating frequency relative to wildtype (FIG. 4B). Streptavidin addition resulted in a heterogenous alteration of the basal gating signature which could not be recapitulated upon streptavidin dosing to OmpG^{wt} (FIGS. 4C and 14B). Altered pore gating signals could be parameterized into three main subtypes. However, there was considerable variation observed between events categorized to each subtype, indicating this subtyping is not exhaustive but was sufficient for the purposes of representing observed signals. Subtype I was dominated by a substantial reduction of gating frequency during the binding event, with a concurrent increase in conductance as exemplified by the representative trace in FIG. 4D. Spontaneous gating was not completely precluded during this state but was reduced in frequency. Subtype II was characterized by high frequency transient gating to ˜75-50% pore unitary conductance but did not demonstrate altered conductance during the bound state, as shown in FIG. 4E. Subtype Ill was also characterized by an increased frequency of gating; however, the frequency and current occlusion was less than that observed in subtype II. Additionally, subtype Ill exhibited an increase in conductance, shown in FIG. 4F. The overall prevalence of binding subtypes was III>II≈I. All subtypes could be ablated upon saturating D-Biotin dosing, demonstrating these signals arose from the specific interaction of streptavidin with the loop 6 displayed SA1.1 motif. The reduction in gating depth and/or frequency upon target interaction likely stems from reduced loop 6 mobility due to the bound analyte, with the other observed ‘spikes’ potentially contributed by the motion of the other dynamic loops sampling the bound analyte near the pore interface. Interestingly, the single analyte resulted in heterogenous yet specific binding signals. It was hypothesized that this could be due to 1) chirality of the tetrameric streptavidin resulting in different surface sampling upon binding, 2) different orientations/conformations of the molecule interacting with the pore upon binding, 3) variable binding poses of the flexible loop within the biotin binding pocket, or 4) a combination of two or more of these possibilities. These possibilities were probed using an engineered monomeric variant; however this protein failed to generate any change in pore behavior. It was hypothesized that this was due to the high degree of molecular selectivity exhibited by OmpG combined with the fact the original SA-1 motif was evolved against streptavidin, the engineered monomeric variant only exhibits ˜56% identity with streptavidin and likely cannot be recognized by the truncated version of the motif.

Following binding signal parameterization, the interevent (τ_{On}) and dwell (τ_{Off}) of streptavidin binding was calculated accounting for all binding subtypes. The average dissociation rate constant (k_{Off}=1/τ_{Off}, mean±SD) of streptavidin binding was 0.783±0.047 s⁻¹ (FIG. 4h) and the average association rate constant (k_{On}=1/[[C*τ_{On}], mean±SD) was 6.914±0.32*10⁴M⁻¹*s⁻¹ (FIG. 4g). Thus, the apparent dissociation constant (K_{d}=k_{Off}/k_{On}) was calculated to be 8.83±0.323 μM (N=3, n=903). This value was over two orders of magnitude higher than was reported for the original un-truncated motif (K_{d}=10 nM) suggesting a substantial loss in affinity stemming from the truncation of the original sequence. However, despite this apparent loss in affinity, the study was able to demonstrate effective detection and quantitation of nanomolar streptavidin within minutes.

In summary, two motifs were identified that demonstrated unique, analyte-specific, and reversible gating signatures arising from direct target interaction with two distinct OmpG display loops.

Multiplex Detection of FG4R and Streptavidin in a Simple Mixture

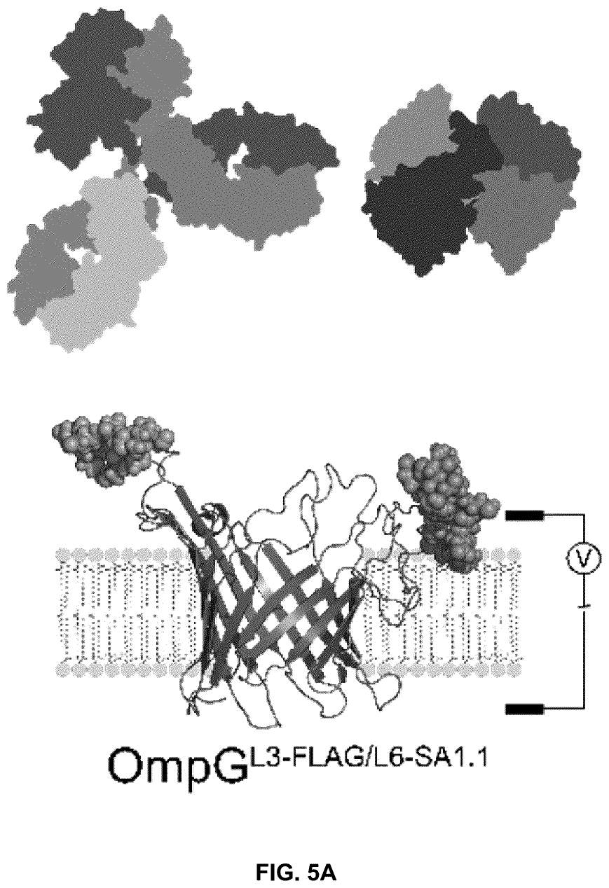

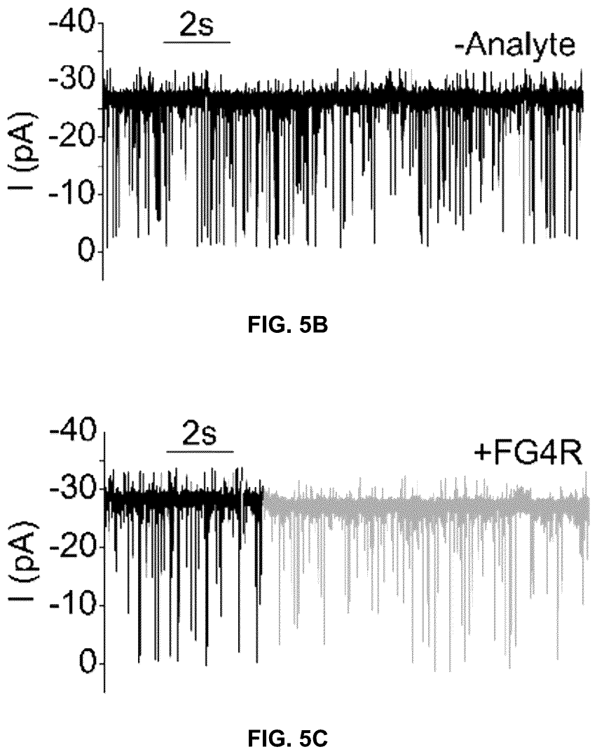

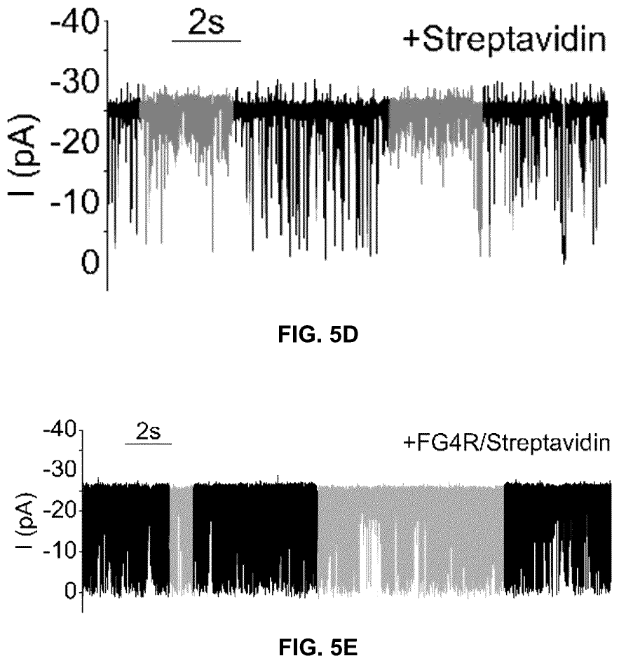

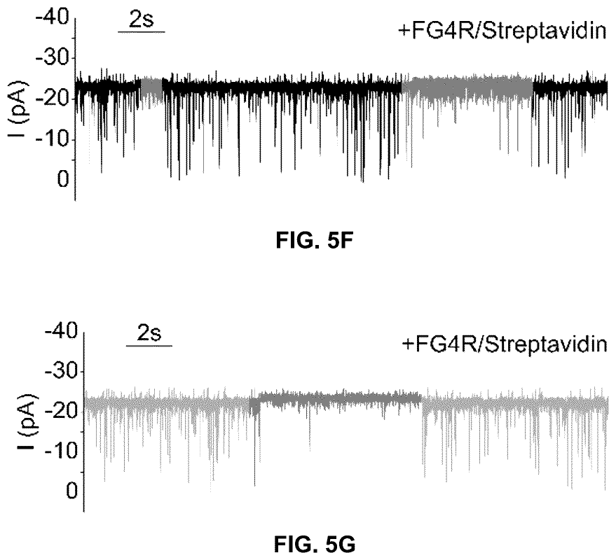

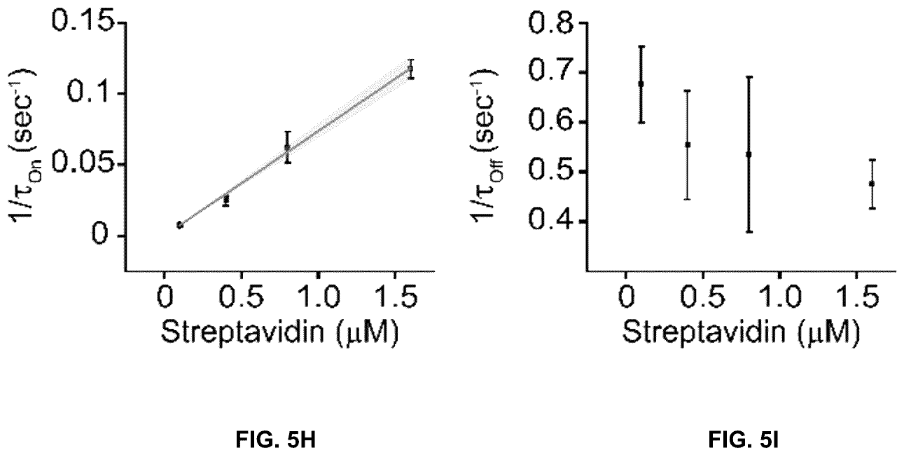

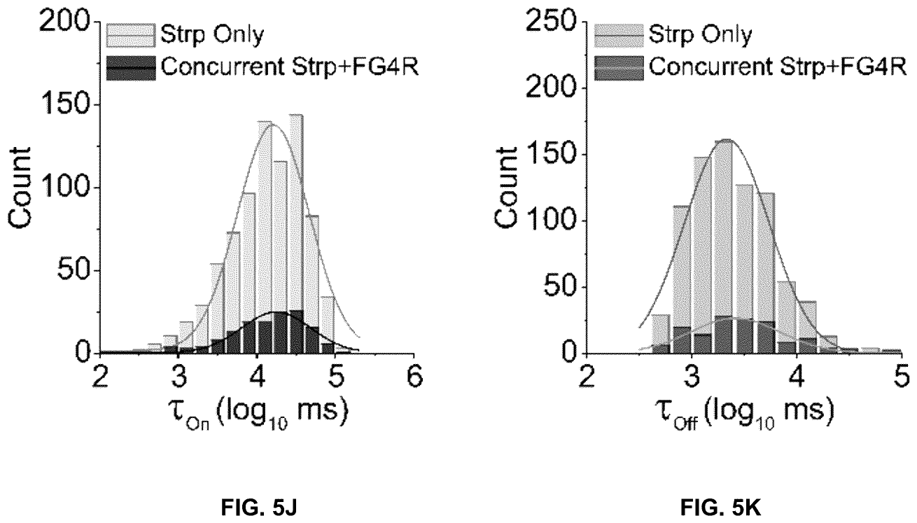

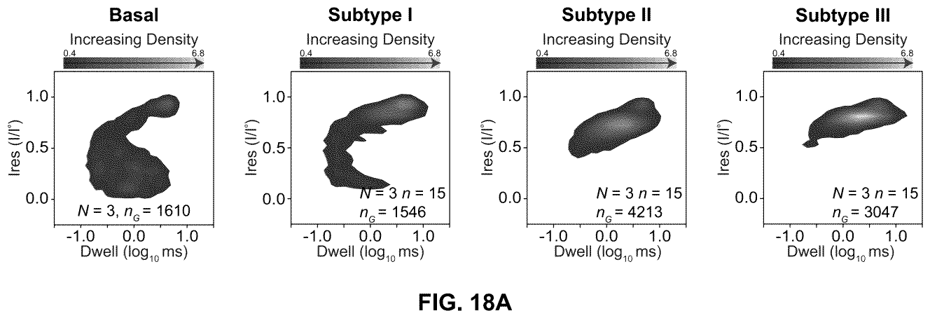

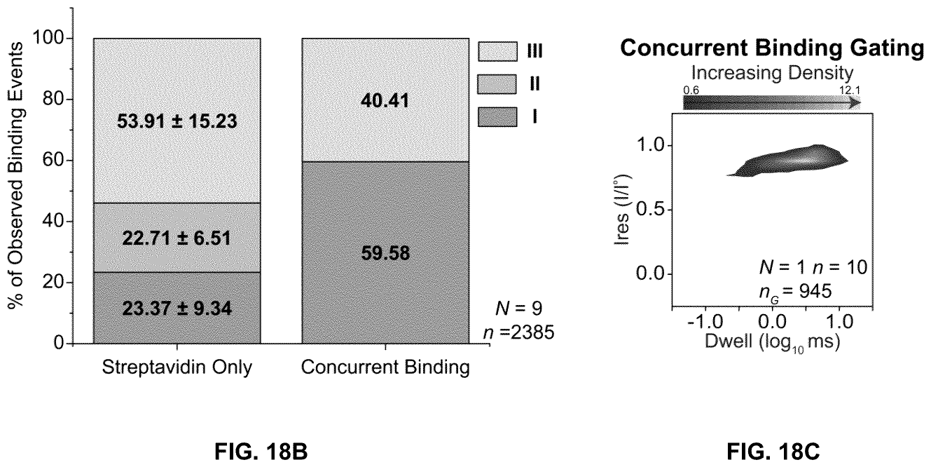

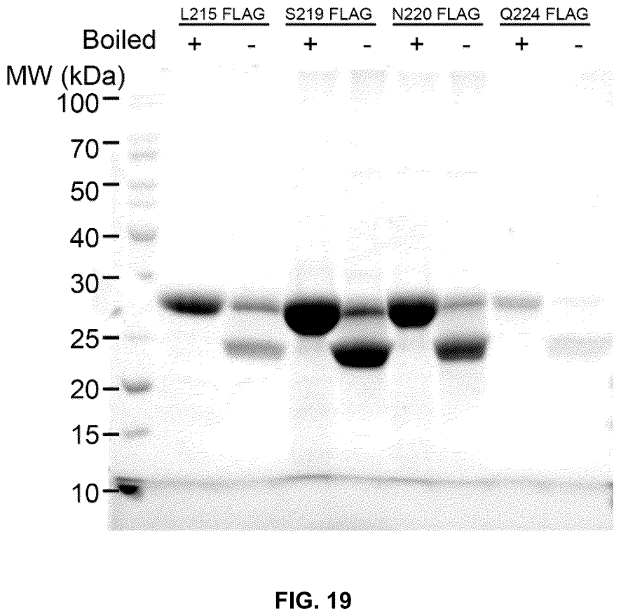

With two motifs (FLAG and SA1.1) demonstrating unique gating signatures arising from direct target interaction with compatible loops, the next goal was to generate a construct for the simultaneous display of these motifs (FIG. 5A). This new multiplex sensor, termed OmpG^{L3-FLAG/L6-SA1.1}, was generated and assessed via single-channel current measurements. In the absence of target the gating profile resembled that observed for OmpG^{L6-SA1.1} suggesting the nature of the motif incorporated into loop 6 dominates the gating profile behavior (FIG. 5B). FG4R (20 nM) addition resulted in recapitulation of the minutes long, reversible reduction in conductance observed with OmpG^{L3-FLAG} sensor (FIG. 5C). Similarly, streptavidin addition (800 nM) recapitulated the heterogenous signal observed with OmpG^{L6-SA1.1} (FIG. 5D). The next goal was to determine if the analyte-specific signals could be recapitulated when present in a simple mixture and so streptavidin (800 nM) and FG4R (20 nM) were dosed to the pore simultaneously. It was observed that gating signature unique to each analyte could be observed individually (FG4R shown in FIG. 5E, streptavidin shown in in FIG. 5F) but most interestingly was that the gating signatures could be observed occurring concurrently (FIG. 5G), indicating both analytes were capable of binding to the pore scaffold without steric hinderance. Interestingly, when assessing the distribution of binding subtypes for streptavidin during concurrent binding with FG4R subtype II was completely ablated, with a concurrent increase in subtypes I & III (FIG. 18).

The binding kinetics interevent (τ_{On}) and dwell (τ_{Off}) of streptavidin with this sensor were then determined when only streptavidin was present using the same method as before for direct comparison to OmpG^{L6-SA1.1}. The average dissociation rate constant (k_{Off}=1/τ_{Off}, mean±SD) was 0.552±0.034 s⁻¹ (N=12, n=2846) and was independent of streptavidin concentration (FIGS. 5I & 15A-15D). The association rate constant, k_{On}, was 7.316±0.259*10⁴M⁻¹s⁻¹ and increased linearly with streptavidin concentration (FIGS. 5H & 15A-15D). These values result in a calculated equilibrium dissociation constant (K_{d}) of streptavidin for the SA1.1 multiplex sensor of 7.544±0.261 μM, in close agreement with the ˜8.8 μM value calculated for OmpG^{L6-SA1.1} suggesting the presence of additional motifs did not perturb this interaction.

The next goal was to determine if concurrent analyte occupancy of the pore altered this kinetic. Due to the significant length of FG4R K_{Off} and the more rapid association kinetics of streptavidin, it was possible to observe numerous streptavidin binding events within each FG4R event, despite the latter's relatively infrequent occurrence, allowing us to use streptavidin's binding kinetics as the model system for concurrent multiplex detection. gaussian fit of τ_{On} and τ_{Off} values were thus compared from log transformed millisecond values representing quantification of streptavidin binding events at 800 nM when only streptavidin is present (N=3, n=813) or considering only streptavidin events concurrent with FG4R binding (N=1, n=146) (FIG. 5j-k). The τ_{On} and τ_{Off} of streptavidin alone was calculated to be 16.204 s and 2.133 s respectively, in comparison to the τ_{On} and τ_{Off} of concurrently-bound streptavidin which were calculated to be 17.573 s and 2.567 s. From this comparison it was determined that there was no significant impact of association (16.204 vs 17.573 s) or dissociation (2.133 vs 2.567 s) kinetics when both analytes were bound to the sensor scaffold, a significant finding as this is the first demonstration of multiplex detection allowing dual occupancy of a nanopore scaffold by two bulky protein analytes. Additionally, the alteration in binding subtype distribution during concurrent occupancy suggests some attributes of the observed subtypes are related to surface-sampling of the bound analyte by OmpG's other loops, as the occupation of loop 3 by FG4R changes the distribution of streptavidin binding signal characteristics but not their overall frequency as would be suggested if the effect were purely positionally based.

From this study it was demonstrated that the binding kinetics of an analyte with its corresponding loop-displayed motif are not significantly impacted by the scaffold hosting other loop-displayed motifs concurrently, suggesting this approach is feasible to pursue with other analytes. The observation of concurrent analyte binding to the sensor was particularly interesting as the prior assumption was the binding of any large protein would preclude binding of any further analytes due to steric occlusion. Overall, OmpG is an attractive malleable scaffold for the development of a nanopore multiplex protein biosensor. This study represents the first multi-functionalization of a single-channel protein nanopore for the direct single-molecule multiplex detection of protein analytes. Prior nanopore multiplex protein detection has been predominantly accomplished using DNA as a capture and carrier molecule, reading the barcoded DNA for detection and quantification of an associated protein biomarker using either a single channel or channel array. These methods consistently achieve higher sensitivity due to the intrinsic capture efficiency of DNA and are expandable based on repertoire of channel functionalization compounds however they lack the molecular recognition resolution of OmpG sensing meaning one can achieve greater discrimination among closely related targets which bind the same affinity reagent, allowing for molecular subtyping not available in other systems. A method for single-channel discrimination of aggregate morphology was achieved using functionalized silicon nitride pore which demonstrated highly generalizable detection parameters but again could not match the selectivity exhibited by OmpG.

Avidity Display of SA1.1 Significantly Impacts Binding Kinetics

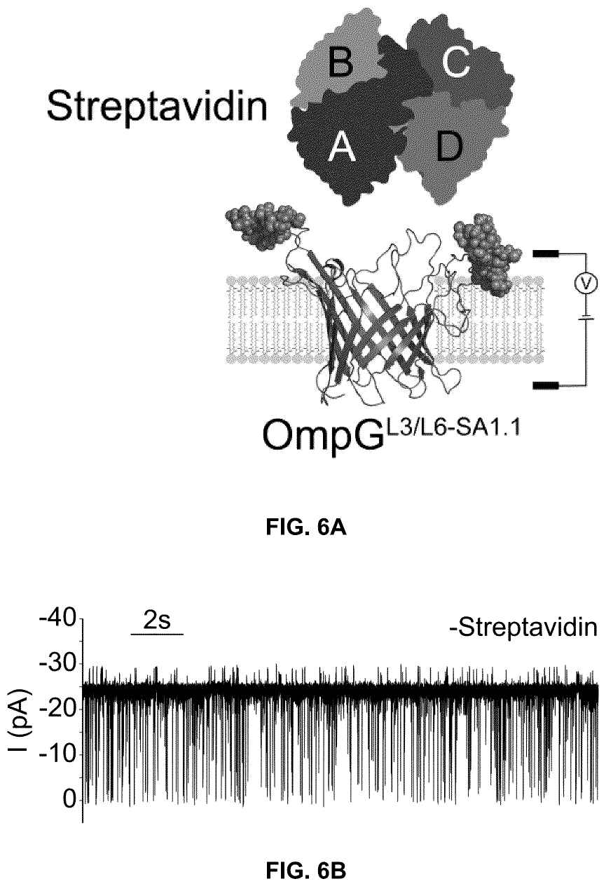

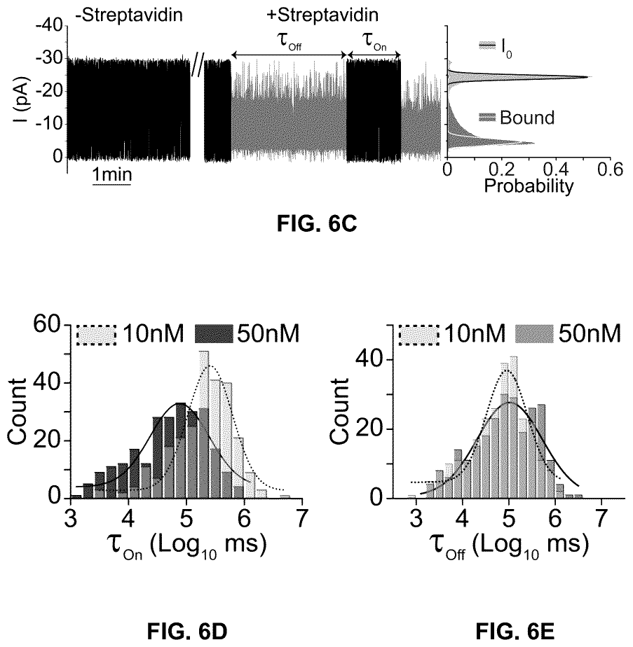

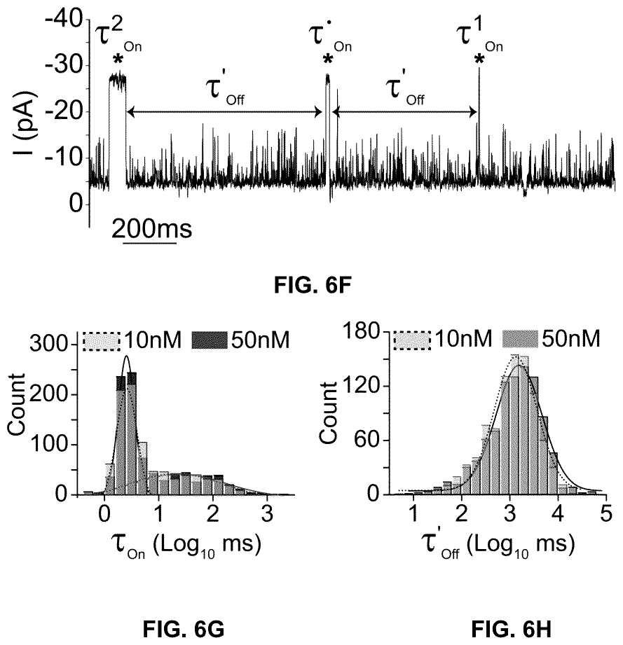

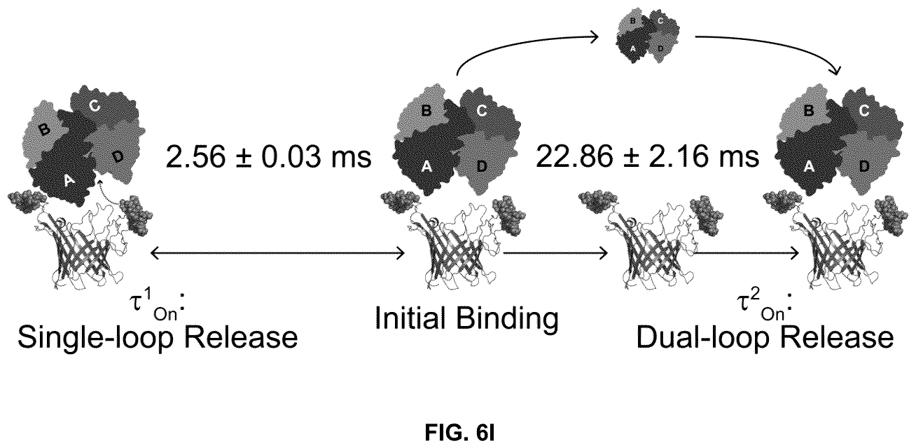

OmpG sensing is stochastic and diffusion mediated, a known limitation in single-molecule sensing, meaning at low analyte concentrations (<nM) detection time becomes inordinately long relative to systems where analytes can be driven to the pore. However, the driving forces typically utilized in these system exploit a general characteristic, such as charge, sacrificing molecular resolution for overall sensitivity. the diffusion limitations of OmpG sensing were addressed via an avidity effect achieved through the display of multiple copies of the same motif on the sensor scaffold, increasing sensitivity while maintaining OmpG's characteristic selectivity. The streptavidin binding motif SA1.1 identified earlier for this study was used due to its moderate affinity and more rapid dissociation kinetics relative to FG4R, increasing the ease in identification and quantification of altered kinetics. A new construct, OmpG^{L3/L6-SA1.1} was generated displaying the SA1.1 motif in both loops 3 and 6 concurrently (FIG. 6A).

In the absence of target OmpG^{L3/L6-SA1.1} basal gating resembled that of OmpG^{L6-SA1.1} and OmpG^{L3-FLAG/L6-SA1.1} (FIG. 6B). Streptavidin addition resulted in a strikingly different signal from that observed in OmpG^{L6-SA1}. This new signal (FIG. 6C) was characterized by a minute(s)-long reduction in current to 27.22±6.31 Iᵣₑₛ (N=5, n=25), with transient high-frequency fluctuations at sub-conductance states between 0.2 and 0.75 Iᵣₑₛ. Streptavidin binding kinetics were starkly different for OmpG^{L3/L6-SA1.1} relative to OmpG^{L6-SA1.1} Streptavidin bound to OmpG^{L6-SA1.1} exhibited an average K_{Off} of 0.783±0.047 s⁻¹ while streptavidin bound to OmpG^{L3/L6-SA1.1} exhibited an average K_{Off} of 0.0103±0.001 s\hat{ }-1, an increase of ˜76× (FIGS. 5I & 6E). However, the calculated K_{On} of OmpG^{L6-SA1.1} was 6.914±0.32*10⁴M⁻¹*s⁻¹ compared with 32.629±8.973*10⁴M⁻¹*s⁻¹ for OmpG^{L3/L6-SA1.1}, an increase of only ˜4.7× (FIGS. 5H & 6D). Upon closer inspection of the new binding signal, there was a gating pattern that can explain this potential discrepancy. Within each minute(s) long binding signal there were numerous transient (1-200 ms) returns to open-pore conductance followed by immediate recapitulation of the low Iᵣₑₛ binding signal (FIG. 6F). This population can also be observed within the all-points histogram in FIG. 6C, showing a small peak at basal conductance observed during streptavidin binding. Assuming the transient returns to basal conductance within this signal represented streptavidin dissociation the K_{Off} of this lower Iᵣₑₛ state was calculated to be 0.701±0.078 s⁻¹, in close agreement with that of OmpG^{L6-SA1.1}, 0.783±0.047 s⁻¹ (FIGS. 5I & 6H). From these observations a model was developed to explain the avidity binding signal.

Each of the minutes long streptavidin signals represents the capture of a unique streptavidin molecule, and as such these events are referred to as ‘discrete’ binding. As streptavidin is a homotetramer it has a valency of 4 relative to potential SA1.1 binding sites, therefore when a single subunit is bound by a SA1.1 copy the other three subunits are available for interaction with the second loop-displayed SA1.1. The ‘discrete’ binding signal observed in this system may reflect the concurrent binding of two SA1.1 copies to the tetramer, holding the molecule over the pore's luminal aperture. Bringing the streptavidin molecule this close to the aperture results in the occlusion of ions through the pore, as characterized by the substantial reduction in conductance. It was further postulated that the rapid gating observed within each of these ‘discrete’ events represents two underlying behaviors. The first behavior, characterized by rapid ‘spikes’ in the low conductance state, can be reasonably interpreted as fluctuations in the position of the bound streptavidin over the aperture or the motion of the other loops non-specifically interacting with the local surface resulting in variable ion flow through the pore. The second behavior, characterized by transient (1-200 ms) occupation of I₀ conductance states following by recapitulation of the prior low Iᵣₑₛ state (FIG. 6F) can be interpreted as the dissociation of one or both SA1.1 copies from the streptavidin tetramer followed by immediate recapture of the same streptavidin molecule by the avidity sensor. The observed bimodal distribution of recapture T_{On} is explained in the model relating to the release of the streptavidin molecule from either a single or both SA1.1 motifs. It was hypothesized that the more rapid recapture rate (2.56±0.003 ms), which is termed τ¹_{On}, reflects the dissociation of a single SA1.1 motif resulting in a rapid return to basal conductance followed by immediate binding. The slower rate (22.86±2.16 ms), termed τ²_{On}, reflects complete analyte dissociation from both motifs and disengagement from the pore lumen, while remaining in the local area. The greater distribution of this population may reflect intrinsic variation within the recapture time or tumbling of the analyte prior to rebinding. A third recapture in FIG. 6F, T_{On}, shows how these two populations overlap meaning some behaviors cannot be effectively categorized due to the limited information contained in the transient returns to basal conductance. From these observations it is proposed that the substantially increased residence on the pore observed for the ‘discrete’ signal is comprised of numerous dissociation and recapture events, the duration of each which is commensurate to a single loop analyte capture. This conclusion is based on 1) the appearance of a new binding state (FIG. 6C) comprised of sub-conductance states (FIG. 6F) with duration commensurate with that of the SA1.1 motif bound state utilized to generate the avidity sensor, 2) the characteristics of the newly observed signal are in agreement with a model where the tetrameric analyte is bridging the loop-displayed motifs, 3) an observed K_{On} for the new state that nears the diffusion limit and is concentration insensitive suggesting analyte recapture, and 4) event concentration dependence for the discrete binding events but not for recapture (FIGS. 5D & 5G). Thus, an increase in analyte recapture efficiency is the underlying mechanism behind the apparent increase in sensor dwell value.

The appearance of this avidity signal and its binding kinetics likely reflects the architecture of motif presentation within our scaffold and streptavidin's valency relative to those motifs. As only an additive effect was observed in the association kinetics, this suggests this strategy may not be feasible for significant increases in sensitivity. The benefit from this strategy appears to be increased analyte residence on the scaffold, this may be due to the unique valency of streptavidin as a target or may be a generalizable feature. If valency is shown to be the driving effect the avidity display of motifs would still have applications in the sensing of other oligomers.

### Conclusions

In summary, this Example demonstrates the multiplex detection of two protein analytes in a simple mixture via protein nanopore. Antibody detection using this system is potentially attractive due to increasing prevalence of recombinant antibody therapeutics necessitating robust and selective assays to quantify therapeutic efficiency.

Materials & Methods

Materials: DH10β and BL21(DE3) E. coli strains and labware were purchased from Thermo Scientific. OverExpress™ C43 E. coli strain was purchased from Lucigen. Phusion® High-Fidelity DNA Polymerase kit, T4 DNA ligase, T4 polynucleotide kinase, and unstained broad range protein standard were purchased from New England BioLabs. DNA Clean & Concentrator kit was purchased from Zymo Research. Miniprep kit was purchased from CoWin Biosciences. Bovine serum albumin, fraction V was purchased from Caisson Labs. Q-Sepharose fast flow resin was purchased from Cytiva. Monoclonal mouse anti-FLAG® antibody FG4R [RRID AB_1957945] and polyclonal goat anti-mouse FITC-conjugated antibody [RRID AB_2533946] were purchased from Thermo Fisher Scientific. Unconjugated streptavidin was purchased from EMD Millipore while streptavidin-Alexa647 was purchased from Jackson ImmunoResearch. n-Octyl-β-D-Glucopyranoside (OG) was purchased from Chem Impex. Potassium chloride (KCl) was purchased from Fisher Scientific. 1,2-Diphytanoyl-sn-glycerol-3-phosphocholine (DPHPC) lipid was purchased from Avanti Polar Lipids. All other reagents were purchased from Research Products International unless otherwise stated.

Cloning of OmpG Constructs: All mutations were produced using oligonucleotides (Eurofins MWG Operon) found in Table 1. Mutagenic polymerase chain reaction (PCR) to introduce loop displayed motif(s) was carried out with the forward and reverse primers oligonucleotides at 500 nM to 0.3 ng/μL pT7/OmpG template for 30× cycles. The resultant PCR mixture product was subjected to DpnI digestion for 3-18 hr at 37° C. to remove template plasmid DNA. Digested PCR mixture was then cleaned using Zymo Clean and & Concentrator kit and eluted in 30 μL 72° C. nuclease-free water. Cleaned PCR product was then transformed into house-made electrocompetent DH103 E. coli under dual ampicillin (150 μg/mL)/streptomycin (50 μg/mL) selection. Colonies containing the desired mutant were confirmed via Sanger sequencing. For constructs to be utilized in single-channel current recordings an additional round of PCR was then carried out to remove the endogenous signal sequence as it would contribute undesired noise to the recording. Signal sequence was removed via oligonucleotide set ‘ssdel’ (Table 1). The PCR mixture product was treated as before and then subjected to a one-pot T4 polynucleotide kinase and ligase reaction to phosphorylate and circularize the DNA for transformation. Ligation mixture was transformed into house-made electrocompetent DH10β as before. Colonies containing the desired deletion were confirmed via Sanger sequencing. All constructs utilized for flow cytometry possess endogenous signal sequence to enable efficient membrane display via native secretory machinery, whereas constructs utilized for single-channel current recordings have had the sequence removed as it is unnecessary for inclusion body purification and will contribute unnecessary noise.

Flow Cytometry: Plasmid(s) containing the engineered OmpG variant of interest were expressed using BL21 (DE3) E. coli. 10 mL 2×YT-Ampicillin[(150 μg/mL]) (Research Products International #X15600) primary cultures for the construct of interest were inoculated using isolated single colonies and allowed to incubate overnight at 30° C./280 rpm. Following overnight growth, the OD600 turbidity of the primary culture was measured and used to normalize the inoculation volume of each 10 mL 2×YT-Ampicillin (150 μg/mL) expression culture. Expression cultures were grown at 30° C. to an optical density (OD₆₀₀) of ˜0.6 at which point expression was initiated via the addition of 500 μM Isopropyl β-D-thiogalactopyranoside (IPTG), cultures were then allowed to incubate overnight at 23° C./280 rpm. Following induction, the OD₆₀₀ turbidity of the induced culture was then measured again to allow for normalization of the number of cells for labeling. A volume representing 4×10⁸ cells was transferred to a sterile, respectively labeled, 2 mL Eppendorf based on the conversion factor of an OD₆₀₀ of 1.0=8×10⁸ cells/mL. All samples for analysis were then pelleted via centrifugation at 3095rcf/4° C./3 min in an Eppendorf 5417R centrifuge using rotor F45-30-11. The media was decanted, and the resultant pellet washed with 1 mL of flow buffer [PBS pH 7.4, 1 mM EDTA, and 0.5% w/v BSA fraction V]. Cells were pelleted again as before and then resuspended in 200 μL of the flow buffer. For constructs displaying the FLAG® epitope motif 16 nM of the anti-FLAG® mouse IgG FG4R antibody [RRID AB_1957945] was added to each 200 μL volume of resuspended cells. For constructs displaying a streptavidin binding motif 800 nM streptavidin-AlexaFluor647 [Jackson ImmunoResearch, 016-600-084] was added to each 200 μL volume of resuspended cells. Mixtures were then incubated at 4° C./15 rpm on a tube revolver for 1 hr, following incubation samples were pelleted and washed as above. For constructs displaying the FLAG® motif an additional labeling step was performed using 3 μL of the goat anti-mouse IgG-FITC solution [RRID AB_2533946]added to each 200 μL volume. Samples were then incubated at 4° C./15 rpm as before. Samples were then pelleted and washed as before. Labeled cells were resuspended in 600 μL of flow buffer and a 50 μL aliquot of the resuspended cells was further diluted into 1 mL of flow buffer for analysis. Samples were then assessed using BD Dual LSRFortessa 5-laser cytometer (BD Biosciences). Analyte binding to the loop-displayed FLAG®-tag was assessed via FITC fluorescence in the ‘FITC’ channel, excitation via 488 nm laser using a 505 nm long pass and 530/30 nm band pass filter, detector voltage set at 500V. Streptavidin binding to the loop-displayed tag was assessed via Alexa647 fluorescence in the ‘APC’ channel, excitation via 640 nm laser using a RG665 nm long pass and 670/30 nm band pass filter, detector voltage set at 725V. Samples run under the ‘low’ flow-rate, averaging ˜15,000-18,000 events per second. Data was log transformed for normalization and analyzed using OriginPro2020b (OriginLab Corporation).

Purification of OmpG Constructs: Plasmid(s) containing the engineered OmpG variant of interest with the signal sequence removed were expressed in C43 E. coli (Lucigen #60452-1) house-made electrocompetent cells. Primary cultures for the construct of interest were inoculated into 10 mL 2×YT-Ampicillin [150 μg/mL] media and allowed to incubate overnight at 30° C./280 rpm. Following overnight growth, the entire primary culture was utilized to inoculate a 300 mL 2×YT Ampicillin [150 ug/mL] culture and allowed to grow at 30° C. to an OD₆₀₀ of 0.5-0.6 at which point expression was initiated via the addition of 500 μM IPTG and the culture was allowed to continue growth at 30° C. for 3-4 hr. Following induction, the culture was collected in a sterile 500 mL Nalgene bottle and harvested via centrifugation in an Eppendorf 5810R centrifuge using rotor A-4-81 at 3184rcf/4° C./20 min. Harvested pellet was lysed in buffer (50 mM Tris-HCL pH 8.0, 1 mM EDTA) via sonication with the following parameters: Misonix instrument, ⅛th inch probe, 30% amplitude, 2 second pulse, 4 second rest, entire cycle repeated twice on ice. The lysate was centrifuged at 20,000rcf/4° C. using an Avanti JXN-26 centrifuge, rotor JA-25.50, and the supernatant was discarded. The inclusion body pellet was resuspended in wash buffer (1.5M urea, 50 mM Tris-HCL pH 8.0) and mixed via stirring for 10-15 min at 23° C. Pellet was obtained again using the same parameters. The inclusion body was then solubilized in binding buffer (8M urea, 50 mM Tris-HCl pH 8.0) with constant stirring for 30 min. Solubilized pellet was then centrifuged as before. Engineered OmpG protein was then purified from resultant supernatant using anion-exchange chromatography under gravity with a 3 mL Q-sepharose bead volume (Cytiva #17051010). Column bound protein was washed with buffers (8M urea, 50 mM Tris-HCL pH 8.0, 75 mM NaCl) and (8M urea, 50 mM Tris-HCL pH 8.0, 200 mM NaCL), and eluted with buffer (8M urea, 50 mM Tris-HCl pH 8.0, 500 mM NaCl). Purity was confirmed via 12% SDS-PAGE gel.

Refolding of OmpG Constructs: Protein concentration was determined from A280 absorbance using nanodrop and extinction coefficient calculated from Benchling web GUI. Denatured OmpG was then diluted with refolding buffer (50 mM Tris-HCl pH 9.0, 114 mM OG) at a 3:5 volume ratio (denatured OmpG: to refolding buffer, final OG concentration 71.3 mM). Protein was incubated at 37° C. for three days-72 hr and refolding efficiency was assessed via house-made 12% SDS-PAGE gel shift assay.

Single-channel recording of OmpG proteins: Single-channel recordings of OmpG were performed similar to previous study. Briefly, experiments were performed in a custom chip apparatus containing two chambers separated by an 25 μm Teflon™ film. An aperture of ˜100 μm diameter was generated in the film via a briefly applied electric arc. The aperture was pretreated with a hexadecane in pentane (10% v/v) solution before the chambers were filled with 0.22 μm filtered recording buffer (50 mM Tris-HCl, pH 6.0, 300 mM KCl). An Ag/AgCl electrode was immersed in each chamber with the cis chamber grounded. 1,2-Diphytanoyl-sn-glycerol-3-phosphocholine (Avanti Polar Lipids, USA) dissolved in pentane (10 mg/mL) was aliquoted to the surface of the buffer in both chambers and monolayers were formed via raising the liquid level up and down across the aperture. Following successful generation of a planar lipid bilayer refolded OmpG proteins (˜500 pM-1 nM, final concentration) were added to the cis chamber. A voltage potential of +250 mV was briefly applied to facilitate OmpG insertion. Following single pore insertion, the applied voltage was reduced to ±50 mV for recording. Current was amplified with an Axopatch 200B integrated patch clamp amplifier (Axon Instruments). Signal was filtered at 2 kHz Bessel when acquired at 10 kHz after digitization with a Digidata 1320A/D board (Axon Instruments). As OmpG can insert bidirectionally pore orientation was determined based on previously reported voltage potential influenced gating behavior by taking an all-points histogram of the entire trace under the applied positive and negative potentials and analyzing its gating pattern. Streptavidin or FG4R was added to the cis or trans chamber depending on pore orientation and the solution was mixed via pipetting a volume representing 1/10th the chamber 25× times. Data was acquired at ‘noisy’ voltage bias (−50 for cis, +50 for trans). Each target concentration studied was acquired from at least three independent pores. Analysis was carried out using Clampfit 11.1 (Molecular Devices). Acquired traces were post filtered digitally using a 500 Hz lowpass gaussian filter. Pore conductance was determined from all-points histogram (bin size 0.025) of a representative 30 sec trace of an independent pore prior to target addition, with extracted counts gaussian fit in OriginPro2020b. Binding events were identified manually, and event start/end times were entered in millisecond values for analysis. When binding kinetics are determined at least 100 bound/unbound events were quantified for each independent pore, extracted values were log₁₀ transformed and used to generate a histogram (0.2 bin size) from which average τ_{On} and τ_{Off} were derived via gaussian fitting using OriginPro2020b. Gating analysis of streptavidin and FG4R binding was performed using MOSAIC. Trace segments constituting behavior of interest were saved as independent .abf files and run using in MOSAIC using the MOSAIC ADEPT 2-State current threshold algorithm under auto settings. Extracted residual current (Iᵣₑₛ, I/I₀) and duration (dwell, ms) of gating events within the I₀ and analyte-bound states were then exported to OriginPro2020b for generation of 2D kernel density contour plots (32×32 grid) detailing gating behavior.

### Example 2: Optimization of an OmpG Nanopore to Enable Single-Molecule Antibody Detection and Subtyping

Results and Discussion

Effect of FLAG Tag Insertion in OmpG Loop 6

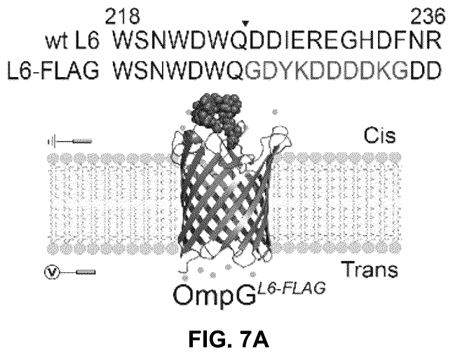

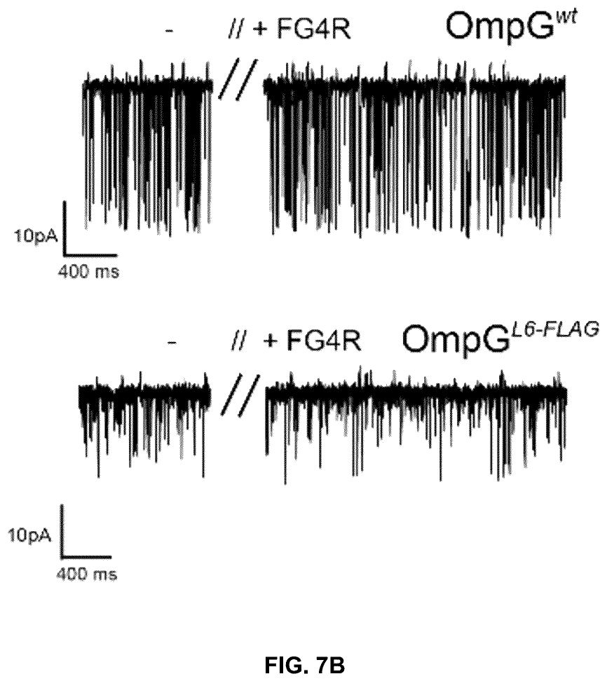

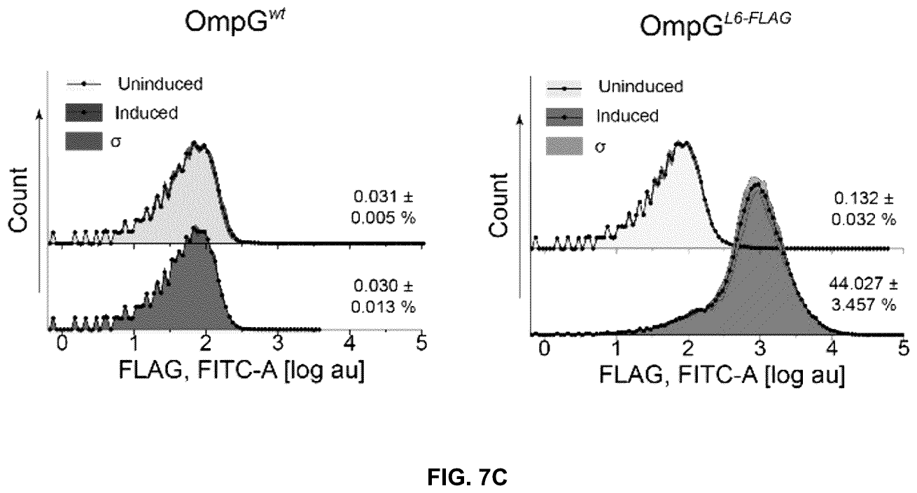

To create a nanopore for detecting FLAG antibody, an OmpG^{L6-FLAG} construct was engineered with the FLAG tag sequence containing two glycine linkers (GDKDDDKG, SEQ ID NO:49) inserted in the loop 6 after residue Q222 (FIG. 7A).

The pore behavior of OmpG^{L6-FL} was then test by single channel recording. OmpG exhibited an open pore current of ˜30 pA with frequent gating spikes (FIG. 7B). Following addition of monoclonal anti-FLAG antibody FG4R, no change in pore behavior was observed (FIG. 7B). OmpG^{L6-FLAG} was also tested under the same conditions. The open-pore current of OmpG^{L6-FLAG} was 30 pA, similar to Omp^{WT}. However, the intensity of gating spikes of OmpG^{L6-FLAG} was shorter than that of OmpG^{wt}, indicating the loop 6 with a FLAG sequence cannot invade to the lumen to fully block the pore. Unexpectedly, the trace after the addition of FG4R to OmpG^{L6-FLAG} did not change either, suggesting that FG4R cannot bind to the FLAG sequence displayed on the loop of OmpG^{L6-FLAG} or the FG4R binding did not trigger any change the current signal (FIG. 7B).

To test the two possibilities, OmpG proteins were expressed at the E. coli outer membrane. Flow cytometry analysis was applied for analysis of FG4R binding to OmpG that displayed the FLAG tag sequence on its loop 6 to the extracellular environment. (Feldhaus, M J., et al. 2003, Nature biotech, Bessette P H., et al., 2004, PEDS, Rollauer S E, et al., 2015, Philos Trans) (Chen, Z Z, et al., 2015, Biosensors and bioelectronics, Mary L T, et al., 1997, FEMS I&MM). As expected, cells transformed with the plasmid encoding OmpG^{WT} did not show FG4R binding under either induced or un-induced condition. In comparison, cells transformed with the plasmid encoding OmpG^{L6-FLAG} exhibited significantly-enhanced fluorescence signals when the expression of OmpG^{L6-FLAG} was induced. This result demonstrated that cells with the FLAG sequence displayed on the OmpG extracellular loop 6 can bind to the FG4R antibody. Thus, failure of the OmpG^{L6-FLAG} nanopore to detect FG4R in the current recording experiments was likely due to that the FLAG tag/FG4R interaction did not trigger any current change signal.

Effect of the Position of FLAG Tag Sequence in OmpG Loop6

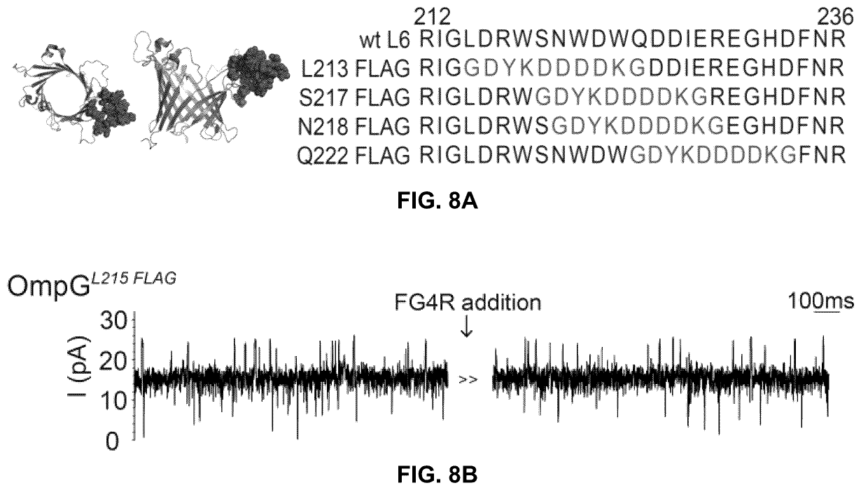

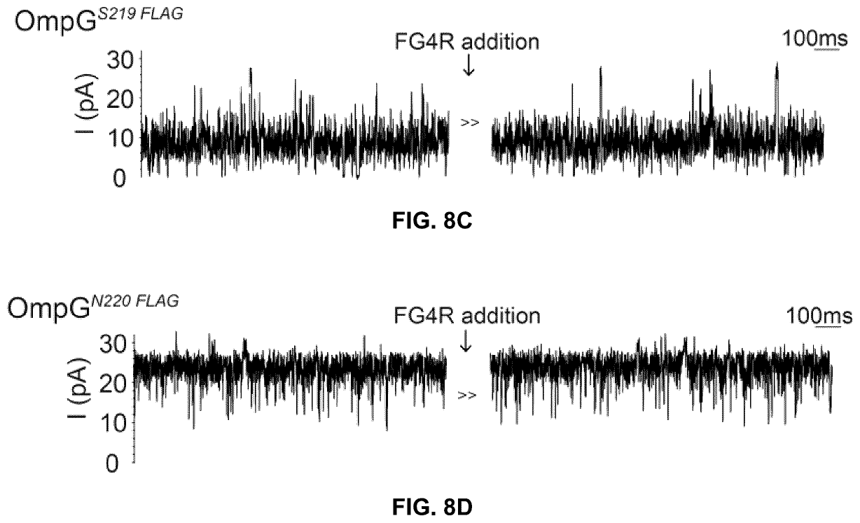

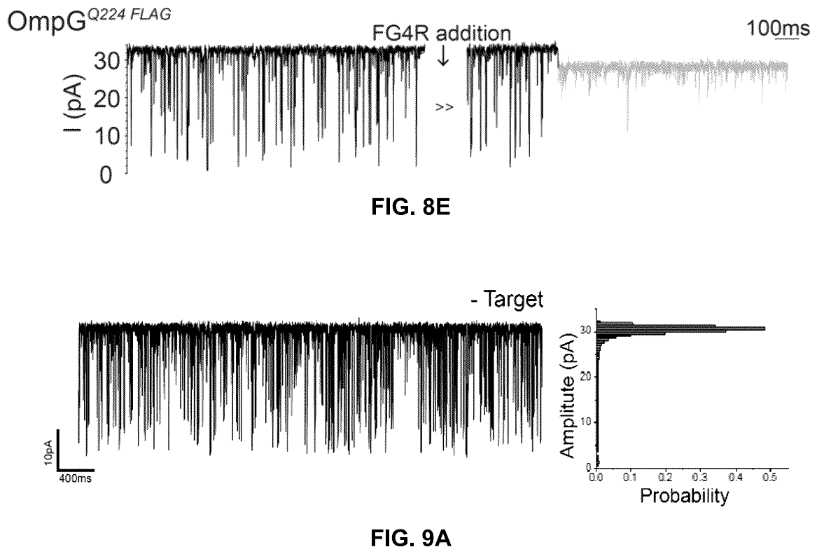

The trace and flow cytometric analysis of OmpG^{L6-FLAG} suggested that new OmpG constructs were required to be engineered. Newly designed four OmpG constructs maintain the loop 6 lengths and replaced the original sequence to FLAG tag at L213, S217, N218, and Q222 (FIG. 8A). After the purification and refolding of each OmpG nanopore, the current was recorded to characterize the pores in the same condition as previously described. Also examined was whether the OmpG nanopore constructs could detect an anti-FLAG monoclonal antibody, FG4R. The open-pore current of OmpG^{L213FLAG} was smaller than that of OmpG^{wt} and have spikes in both opening and closing states, meaning a rapid conductance change due to the fluctuation of the loops of OmpG. This pore characteristic did not change after 30 nM target FG4R indicating that there is no binding interaction that can be observed by OmpG^{L213FLAG} (FIG. 8B). The single channel current recording of OmpG^{S217FLAG} showed similar characteristics with that of OmpG^{L213FLAG}, but with greater fluctuation in the unitary conductance. This construct also didn't have any binding signal even if 30 nM FG4R was added (FIG. 8c). The single channel current measurement of OmpG^{N218FLAG} showed short spikes having half-closing states and did not show the FG4R binding event. Lastly, however, the OmpG^{Q222-FLAG} construct generated a similar current trace with that of OmpG^{WT} having spikes toward closing states. Target FG4R was added into the chamber with the same method as others, and OmpG^{Q222-FLAG} showed binding signals of 10% reduced current flow (FIG. 8e). Based on the results as shown in the FIG. 8, the FLAG binding motif presentation within the loop 6 of OmpG plays a role in antibody detection.

Detection of FLAG Monoclonal Antibodies Using OmpG^{Q222-FLAG}

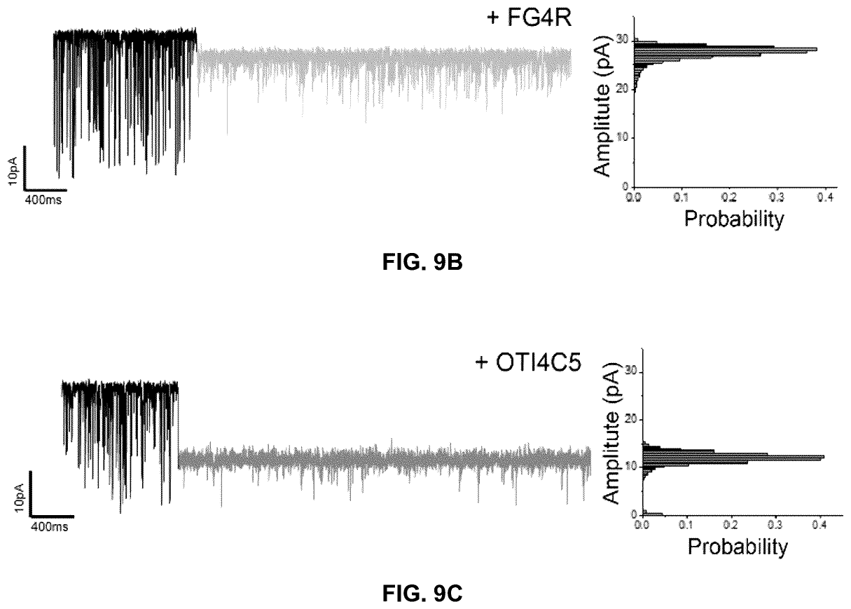

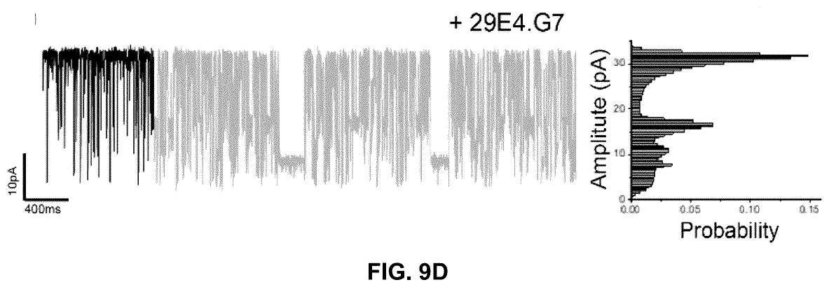

OmpG^{Q222-FLAG} was have further studied with three different FLAG antibody clones, FG4R, OTI4C5 and 29E4.G7 to examine whether OmpG^{Q222-FLAG} can differentiate each monoclonal antibody. Adding different types of monoclonal antibodies into the FLAG binding scaffold containing loop facing chamber generated different binding signals. Same as previous current recordings, each monoclonal subtype was tested under 50 mV, and all pores used to test for antibody subtyping were the size of 30 pA. The antibody bound states were described as following explanation (FIG. 9). Compared to the current trace of before target addition (FIG. 9A), the fully opened current of antibody clone FG4R bound signal was 2-3 pA decreased amplitude. Also, the gating impulses observed during binding were to a higher Ires level than that of unbound recording and were reduced in frequency (FIG. 9B). The binding signal of FLAG clone OTI4C5 exhibited a 20 pA reduction in current and the frequency of the spike was less than that of in a trace before target addition (FIG. 9C). Lastly, the bound signal of FLAG clone 29E4.G7 displayed fluctuating gating patterns so that the baseline was hard to be observed (FIG. 9D). As shown in the current traces and histograms in FIG. 9, it was possible to clearly detect the different anti-FLAG monoclonal antibodies and each clone generated distinct binding signals.

Differentiation of FLAG Antibody Species within a Mixture Using OmpG^{Q222-FLAG}

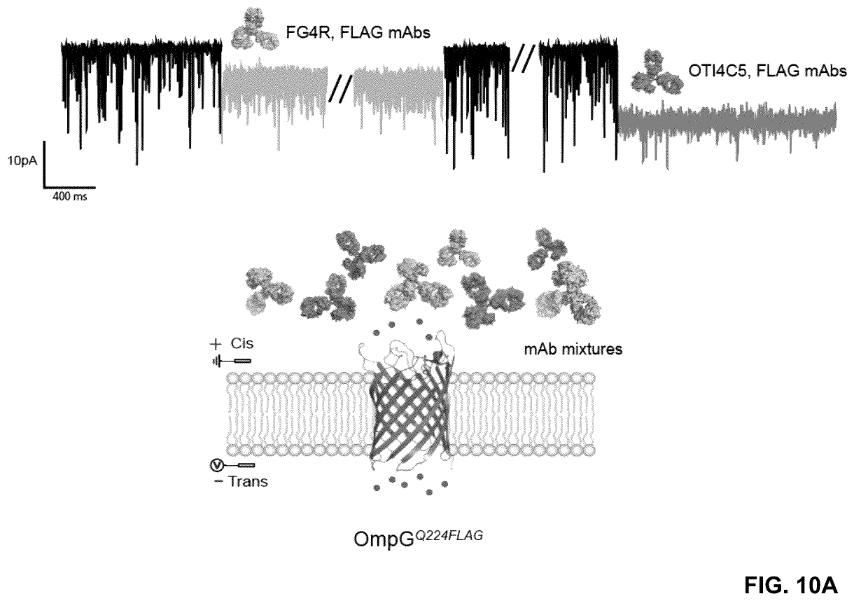

The OmpG^{Q222-FLAG} can not only detect the anti-FLAG antibody but also differentiate antibody clones. As a further study, two monoclonal antibodies FG4R and OTI4C5 were mixed in a 1:1 ratio and added to the chamber for 60 nM to see if OmpG^{Q222-FLAG} can detect and differentiate two different antibodies in a mixture. It was possible to observe recapitulation of the previously observed signals when both analytes were present in a simple mixture during the 8 hours of recording (FIG. 10A). Next, tested was anti-FLAG polyclonal antibodies to OmpG^{Q222-FLAG}. The same current recording protocol was used, and 20 nM of polyclonal antibody was added. During 15 hours of recording, three different types of the binding signal were generated meaning at least three different subtypes of antibody exist in the polyclonal antibody.

### Conclusions

By adjusting the presentation of FLAG binding motif on the loop6, an OmpG nanopore construct was have created that can detect and discriminate different anti-FLAG mAbs and pAbs in mixture. Notably, the off-rate of the antigen-antibody binding can be easily derived from the dwell time of the binding events. Thus, this work points out the feasibility of applying OmpG nanopore sensor for simultaneous screening, selection and validation of the efficacy of mAbs from hybridoma supernatant. While the present current recording platform is not compatible with 96-well plate reading, a needle-shaped nanopore sensor platform could be developed to operate in plate-based dip-and-read configuration for high throughput screening.

Material and Methods

Cloning of OmpG constructs. All modified OmpG constructs were generated by using pT7-OmpG wild-type as the template. Overlapping mutagenesis PCRs were performed using primers containing the FLAG tag sequence (Eurofins genomics) (Table 2). For the OmpG Loop6 FLAG insertion, primers are designed to insert a FLAG binding motif with a glycine at each end (GDYKDDDKG, SEQ ID NO:50) after Q222 in the sequence of loop 6. OmpG loop6 FLAG replacements are designed by replacement of 9 amino acids from L213, S217, N218 and Q222 each (GDYKDDDKG, SEQ ID NO:51). The PCR products were subjected to DpnI (New England Biolabs) digestion at 37° C. overnight to remove the template plasmids and transformed into house-made chemically competent DH10p cells. Plasmids containing mutated genes were isolated and confirmed by Sanger sequencing (Eurofin genomics).

+0 TABLE 2
List of oligonucleotides to generate OmpGFLAG
mutations. Inserted sequences were underlined.

| Generated | |
|---|---|
| OmpG | |
| construct | Oligonucleotide Sequence |
| OmpG^{L6-FLAG} | Forward: |
| | 5′-TAAAGATGACGATGATAAAG |
| | ATGATATTGAACGTG-3′ |
| | (SEQ ID NO: 52) |
| | Reverse: |
| | 5′-TCATCGTCATCTTTATAATC |
| | CTGCCAGTCCCAGTTAC-3′ |
| | (SEQ ID NO: 53) |
| OmpG^{L215 FLAG} | Forward: |
| | 5′-TTATAAAGATGACGATGATA |
| | AAGGAGATGATATTGAACGT |
| | G-3′ |
| | (SEQ ID NO: 54) |
| | Reverse: |
| | 5′-TCGTCATCTTTATAATCTCC |
| | CCCAATGCGCGTATAC-3′ |
| | (SEQ ID NO: 55) |
| OmpG^{S219 FLAG} | Forward: |
| | 5′-AAAGATGACGATGATAAAGG |
| | ACGTGAAGGCCATGATTTTA |
| | AC-3′ |
| | (SEQ ID NO: 56) |
| | Reverse: |
| | 5′-TATCATCGTCATCTTTATAA |
| | TCTCCCCAGCGATCCAGCCC |
| | -3′ |
| | (SEQ ID NO: 57) |
| OmpG^{N220 FLAG} | Forward: |
| | 5′-AAAGATGACGATGATAAAGG |
| | AGAAGGCCATGATTTTAAC-3′ |
| | (SEQ ID NO: 58) |
| | Reverse: |
| | 5′-ATCATCGTCATCTTTATAAT |
| | CTCCACTCCAGCGATCCAG-3′ |
| | (SEQ ID NO: 59) |
| OmpGQ^{224 FLAG} | Forward: |
| | 5′-TATAAAGATGACGATGATAA |
| | AGGATTTAACCGTGTAGGTT |
| | TATTTTAC-3′ |
| | (SEQ ID NO: 60) |
| | Reverse: |
| | 5′-ATCGTCATCTTTATAATCTC |
| | CCCAGTCCCAGTTACTC-3′ |
| | (SEQ ID NO: 61) |

Preparation of OmpG nanopore constructs. Expression and purification of OmpG nanopore constructs were performed by following the previous protocol with slight modification (Fahie, M et al, 2021, ACS sensors). The generated OmpG L213, S217, N218, Q222 FLAG ts were transformed into chemically competent E. coli BL21 (DE3) cells and inoculated into 1000 mL of 2×YT media (Research Product International) containing 150 μg/mL ampicillin (Research Product International), and incubated at 37° C. with 250 rpm. When the optical density (OD₆₀₀) reached 0.5-0.6, the final concentration of 0.5 mM IPTG was added to initiate point expression and the culture was incubated at 16° C. for 16 hours with constant shaking at 250 rpm. Cells were harvested by using a centrifuge (Eppendorf 5810R) at 4° C., 3184 rcf for 20 minutes. The pellets were resuspended with buffer (50 mM Tris-HCl pH 8.0, 1 mM EDTA), and then sonicated on ice for 14 minutes. The lysate was centrifuged with Avanti JXN-26 centrifuge at 4° C. with 20,000 rcf for 20 minutes, and the supernatant was discarded. The OmpG containing pellet was resuspended with 50 mM Tris-HCl pH 8.0, 1.5 M Urea containing buffer and incubated at 23° C. for 15 minutes with continuous stirring using a magnetic bar. The dissolved inclusion body pellet was then centrifuged at 4° C. with 20,000 rcf for 20 minutes. The collected pellet was incubated with constant stirring in denaturation buffer (50 mM Tris-HCl pH 8.0, 8 M UREA) at 23° C. for 30 minutes, then centrifuged the inclusion lysate using the same condition. After 20 minutes of centrifugation, the supernatant was loaded on the anion exchange gravity column containing Q Sepharose fast flow beads (Cytiva). The protein-loaded beads were washed using wash buffer (50 mM Tris-HCl pH 8.0, 8 M UREA and 75 mM NaCl), and the protein was eluted by 50 mM Tris-HCl pH 8.0, 8 M UREA and 200 mM NaCl. Each fraction was collected and loaded on a 12% SDS-PAGE gel to check the purity of the protein. The purified OmpG proteins were refolded by mixing with refolding buffer (110 mM Octyl-glucoside and 50 mM Tris-HCl pH 9.0) in a 3:8 volume ratio and incubated at 37° C. for three days. Refolding efficiency was tested with a 12% SDS-PAGE by comparing the mobility of refolded OmpG with the heated refolded OmpG (FIG. 18). Refolded OmpG proteins were aliquoted and stored with 20% glycerol at −80° C. until further usage.

Ensemble measurement of engineered OmpG constructs using flow cytometry. Plasmid(s) containing the engineered OmpG variant of interest were transformed into house-made electrocompetent BL21 A1 E. coli under dual kanamycin (50 μg/mL)/tetracycline (10 μg/mL) selection. Primary cultures consisting of a 10 mL volume of 2×YT-kanamycin/tetracycline were inoculated via the addition of an isolated colony and incubated overnight at 30° C./280 rpm. OD₆₀₀ turbidity measurement of the primary cultures was taken following overnight growth and used to normalize the inoculation volume for secondary 10 mL 2×YT-kanamycin/tetracycline expression cultures. Expression cultures were grown at 30° C. until an OD₆₀₀ of ˜0.6 at which point 100 μM L-arabinose was added to initiate expression, cultures were then allowed to continue incubating at 30° C. for an additional 2 hour. After expression completed the OD₆₀₀ was measured again for normalization of the number of cells for labeling. Based on the conversion factor of an OD₆₀₀ of 1.0 equaling 8×10⁸ cell/mL, aliquot a volume of the respective culture representing 4×10⁸ cells into a corresponding sterile 2 mL Eppendorf tube. Cells were then pelleted via centrifugation at 3095rcf/4° C. for 3 minutes. The supernatant of media was discarded, and the cells were washed using 1 mL flow buffer (1×PBS pH 7.4, 1 mM EDTA, 0.5% w/v BSA). Cells were collected again using the same centrifuge parameters. Supernatant was again discarded, and the washed cells resuspended in 200 μL flow buffer. At this time the anti-FLAG mouse antibody FG4R [AB_1957945] was added at a final concentration of 16.5 nM. Cell/antibody mixture was then allowed to incubate at 4° C./15 rpm on a tube revolver for 1 hour. Following primary labeling cells were collected and washed as before, 3 μl of the secondary antibody, goat anti-mouse polyclonal-FITC [AB_2533946], was added to 200 μl of second resuspension, and the cell/antibody mixture was then allowed to incubate at 4° C./15 rpm on a tube revolver for 1 hour. Following secondary labeling cells were collected and washed as before. Labeled cells were resuspended in 600 μL of flow buffer and a 50 μL aliquot of the resuspended cells was further diluted into 1 mL of flow buffer for analysis. Samples were then assessed using BD Dual LSRFortessa 5-laser cytometer. Analyte binding to the loop-displayed FLAG-tag was assessed via FITC fluorescence in the ‘FITC’ channel, excitation via 488 nm laser using a 505 nm long pass and 530/30 nm band pass filter, detector voltage set at 500V. Samples run under the ‘low’ flow-rate, averaging ˜15,000-18,000 events per second.

Current recording of OmpG nanopores and data analysis. To record the channel characteristics of OmpG nanopores, an apparatus having two chambers divided by a Teflon film with a thickness of 25 μm was prepared. This Teflon film has an aperture of ˜100 μm diameter made by a sharply applied electric arc, was placed between the two chambers, and was pretreated with a 10% (v/v) hexadecane/pentane solution. Two chambers were filled with a 0.22 μm filtered recording buffer (300 mM KCl and 50 mM Na₂HPo₄, pH 6.0), and 1,2-Diphytanoyl-sn-glycerol-3-phosphocholine (DPhPC) (Avanti Polar Lipids, USA) dissolved in pentane (10 mg/mL, m/v) was added to both chambers to make a lipid bilayer. Electrodes made by Ag/AgCl were placed into each chamber, and a lipid bilayer was formed by pipetting up and down the buffers so that lipid can be towards to aperture and form a bilayer. A molecular device Axopatch 200B (Axon Instruments), a patch-clamp amplifier, was used to adjust the applied voltage and amplify the ionic current flow through a lumen of the OmpG nanopore. A digitized current signal by a Digidata 1320A/D board (Axon Instrument) was filtered by 2 kHz Bessel. When the bilayer became stable, OmpG proteins were added to the cis chamber (final OmpG concentration is less than 1 nM) and 250 mV was applied to facilitate the insertion of OmpG into the bilayer. When a single pore was inserted, reduced the voltage to 50 mV, recorded the current flow and determined the pore orientation by the histogram of the current flow of the whole trace. Then, 30 nM of FLAG antibody was added into the chamber where the loops were located and was mixed thoroughly via pipetting. The used antibodies were clone FG4R (Thermo fisher Scientific), OTI4C5 (OriGene Technologies), 29E4.G7 (Rockland), and Polyclonal (Proteintech). The recorded traces were analyzed in Clampfit 11.2 using a single channel search after the filtration by a 500 Hz low-pass Bessel filter. The binding signals were manually analyzed through comparisons of the trace before and after adding the target antibodies. The conductance of the pore before and after target addition was determined by all-point histogram having a bin size 0.025, and the histogram was gaussian fitted in OriginPro 2021.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

What is claimed is:

1. A composition comprising a nanopore disposed in a membrane preparation, wherein the nanopore comprises an outer membrane protein G (OmpG) of E. coli origin having fourteen β-strands connected by seven flexible loops on a first side of the membrane preparation and seven short turns on a second side of the membrane preparation, wherein a heterologous peptide is inserted within one or more of the flexible loops.

2. The composition of claim 1, wherein the heterologous peptide is inserted between two amino acids within the flexible loops.

3. The composition of claim 2, wherein the heterologous peptide replaces one or more of the amino acids within the flexible loops.

4. The composition of claim 3, wherein the number of amino acids replaced is the same as the number of amino acids in the heterologous peptide.

5. The composition of claim 1, wherein the OmpG comprises the amino acids sequence SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9, or a variant thereof having at least 80%, 85%, 90%, 95%, 96%, 97%, 98%, 99%, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, or SEQ ID NO:9.

6. The composition of claim 1, wherein the OmpG comprises the amino acids sequence SEQ ID NO:1, and wherein loop 1 comprises amino acids E16 to A32, loop 2 comprises amino acids Q52 to D68, loop 3 comprises amino acids G96 to N109, loop 4 comprises amino acids F138 to T150, loop 5 comprises amino acids E175 to I194, loop 6 comprises amino acids R212 to R236, and loop 7 comprises amino acids E258 to V275 of SEQ ID NO:1.

7. The composition of claim 6, wherein the heterologous peptide is inserted within, replaces, or a combination thereof, amino acids E16 to A31 of SEQ ID NO:1.

8. The composition of claim 7, wherein the heterologous peptide replaces 1, 2, 3, 4, 5, 6, 7, 8, 9, 10, 11, 12, 13, 14, 15, or 16 contiguous amino acids selected from E16, I17, E18, N19, V20, E21, G22, Y23, G24, E25, D26, M27, D28, G29, L30 and A31.

9. The composition of claim 8, wherein the heterologous peptide is inserted within, replaces, or a combination thereof, amino acids Q52 to D68 of SEQ ID NO:1.

10. The composition of claim 9, wherein the heterologous peptide replaces 1, 2, 3, 4, 5, 6, 7, 8, 9, 10, 11, 12, 13, 14, 15, or 16 contiguous amino acids selected from Q52, E53, G54, P55, V56, D57, Y58, S59, A60, G61, K62, R63, G64, T65, W66, F67, and D68.

11. The composition of claim 10, wherein the heterologous peptide is inserted within, replaces, or a combination thereof, amino acids G96 to N109 of SEQ ID NO:1.

12. The composition of claim 11, wherein the heterologous peptide replaces 1, 2, 3, 4, 5, 6, 7, 8, 9, 10, 11, 12, 13, 14, 15, or 16 contiguous amino acids selected from G96, Y97, H98, Y99, V100, D101, E102, P103, G104, K105, D106, T107, A108 and N109.

13. The composition of claim 12, wherein the heterologous peptide is inserted within, replaces, or a combination thereof, amino acids F138 to T150 of SEQ ID NO:1.

14. The composition of claim 13, wherein the heterologous peptide replaces 1, 2, 3, 4, 5, 6, 7, 8, 9, 10, 11, 12, 13, 14, 15, or 16 contiguous amino acids selected from F138, A139, N140, D141, L142, N143, T144, T145, G146, Y147, A158, D149 and T150.

15. The composition of claim 14, wherein the heterologous peptide is inserted within, replaces, or a combination thereof, amino acids E175 to I194 of SEQ ID NO:1.

16. The composition of claim 15, wherein the heterologous peptide replaces 1, 2, 3, 4, 5, 6, 7, 8, 9, 10, 11, 12, 13, 14, 15, or 16 contiguous amino acids selected from E175, R176, G177, F178, N179, M180, D181, D182, S183, R184, N185, N186, G187, E188, F189, S190, T191, Q192, E193 and I184.

17. The composition of claim 16, wherein the heterologous peptide is inserted within, replaces, or a combination thereof, amino acids L213 to D228 of SEQ ID NO:1. Therefore, in some embodiments, the heterologous peptide is inserted after L215, D216, R217, W218, S219, N220, W221, D222, W223, Q224, D225, D226, I227, E228, R229, E230, G231, H232, or D233.

18. The composition of claim 17, wherein the heterologous peptide replaces 1, 2, 3, 4, 5, 6, 7, 8, 9, 10, 11, 12, 13, 14, 15, or 16 contiguous amino acids selected from L213, D214, R215, W216, D217, W218, Q219, D220, D221, I222, E223, R224, E225, G226, H227, D228, F229, H230 and R231.

19. The composition of claim 1, comprising 2, 3, 4, 5, 6, or 7 distinct heterologous peptides independently inserted within the one or more flexible loops.

20. The composition of claim 1, wherein the membrane preparation comprises a planar lipid bilayer.

21. The composition of claim 20, wherein the membrane preparation comprises a micelle, a bacterium, or a eukaryotic cell.

22. A method of detecting binding of a ligand to a target, the method comprising:
exposing the composition of claim 1 to a target;
assessing a gating pattern of the nanopore; and
detecting binding of the target to the heterologous peptide based on the gating pattern.

23. The method of claim 22, wherein the target is a protein, a virus, a bacteria, a nucleic acid, or a mammalian cell.

24. The method of claim 22, wherein the target is an antibody or hybridoma.
